SEQUENCE LISTING (3 sequences)

SEQ ID NO: 1 - DNA, 20 bp, Artificial Sequence
  Description of Artificial Sequence Synthetic primer
aagaaaaaccccgcctgtaa

SEQ ID NO: 2 - DNA, 23 bp, Artificial Sequence
  Description of Artificial Sequence Synthetic primer
cctgttctgactactgcctctcc

SEQ ID NO: 3 - DNA, 27 bp, Artificial Sequence
  Description of Artificial Sequence Synthetic oligonucleotide
cctgatgtgatgttctccatgttcagc

(19) United States
(12) Patent Application Publication
(10) Pub. No.: US 2023/0144399 A1
(43) Pub. Date: May 11, 2023

(54) QUINOLINE COMPOUNDS FOR THE TREATMENT AND PROPHYLAXIS OF HEPATITIS B VIRUS DISEASE

(71) Applicant: Hoffmann-La Roche Inc., Little Falls, NJ (US)

(72) Inventors: Song FENG, Shanghai (CN); Zongxing QIU, Shanghai (CN); Guolong WU, Shanghai (CN); Dongdong CHEN, Shanghai (CN); Chao LI, Shanghai (CN)

(73) Assignee: Hoffmann-La Roche Inc., Little Falls, NJ (US)

(21) Appl. No.: 16/955,335

(22) PCT Filed: Dec. 14, 2018
(86) PCT No.: PCT/EP2018/084868

§ 371 Date: Jun. 18, 2020

(30) Foreign Application Priority Data
Dec. 19, 2017 (CN) PCT/CN2017/117285
Oct. 9, 2018 (CN) PCT/CN2018/109424

(51) Int. Cl. C07D 215/18; C07D 215/52; C07D 215/233; C07D 215/20; A61P 31/20; C07D 215/14

(52) U.S. Cl. CPC C07D 215/18; C07D 215/52; C07D 215/233; C07D 215/20; A61P 31/20; C07D 215/14

(57) ABSTRACT

The present invention provides novel compounds having the general formula: wherein R¹ to R¹¹ are as described herein, compositions including the compounds and methods of using the compounds.

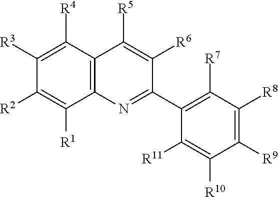

The present invention relates to organic compounds useful for therapy and/or prophylaxis of HBV infection in a mammal, and in particular to HBV cccDNA (covalently closed circular DNA) inhibitors useful for treating HBV infection.

### FIELD OF THE INVENTION

The present invention relates to novel quinoline derivatives having pharmaceutical activity, their manufacture, pharmaceutical compositions containing them and their potential use as medicaments.

The present invention relates to compounds of formula (I)

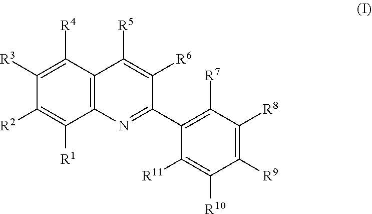

wherein R¹ to R¹¹ are as described below, or a pharmaceutically acceptable salt thereof.

Hepatitis B virus (HBV) infection is one of the most prevalent viral infections and is a leading cause of chronic hepatitis. It is estimated that worldwide, around 2 billion people have evidence of past or present infection with HBV. Over 250 million individuals are currently chronically infected with HBV and are therefore at high risk to develop liver fibrosis, cirrhosis and hepatocellular carcinoma (HCC). There are data to indicate ˜800,000 deaths per year are directly linked to HBV infection (Lozano, R. et al., Lancet (2012), 380 (9859), 2095-2128; Goldstein, S. T. et al., Int J Epidemiol (2005), 34 (6), 1329-1339).

Many countries in the world administer hepatitis B vaccine starting at birth or in early childhood, which has greatly reduced the incidence and prevalence of hepatitis B in most endemic regions over the past few decades. However, the vaccine has no impact on people who were infected before the widely use of the vaccine in developing end-stage liver disease or HCC (Chen, D. S., J Hepatol (2009), 50 (4), 805-816). Vaccination at birth of infants born to HBV positive mothers is usually not sufficient for protecting vertical transmission and combination with hepatitis B immune globulin is needed (Li, X. M. et al., World J Gastroenterol (2003), 9 (7), 1501-1503).

Currently FDA-approved treatments for chronic hepatitis B include two type 1 interferons (IFN) which are IFNalfa-2b and pegylated IFN alfa-2a and six nucleos(t)ide analogues (NAs) which are lamivudine (3TC), tenofovir disoproxil fumarate (TDF), adefovir (ADV), telbivudine (LdT), entecavir (ETV), and vemlidy (tenofovir alafenamide (TAF)). IFN treatment is finite, but it is known to have severe side effects, and only a small percentage of patients showed a sustained virological response, measured as loss of hepatitis B surface antigen (HBsAg). NAs are inhibitors of the HBV reverse transcriptase, profoundly reduce the viral load in vast majority of treated patients, and lead to improvement of liver function and reduced incidence of liver failure and hepatocellular carcinoma. However, the treatment of NAs is infinite (Ahmed, M. et al., Drug Discov Today (2015), 20 (5), 548-561; Zoulim, F. and Locarnini, S., Gastroenterology (2009), 137 (5), 1593-1608 e1591-1592).

HBV chronic infection is caused by persistence of covalently closed circular (ccc)DNA, which exists as an episomal form in hepatocyte nuclei. cccDNA serves as the template for viral RNA transcription and subsequent viral DNA generation. Only a few copies of cccDNA per liver cell can establish or re-initiate viral replication. Therefore, a complete cure of chronic hepatitis B will require elimination of cccDNA or permanently silencing of cccDNA. However, cccDNA is intrinsically very stable and currently available therapeutics could not eliminate cccDNA or permanently silence cccDNA (Nassal, M., Gut (2015), 64 (12), 1972-1984; Gish, R. G. et al., Antiviral Res (2015), 121, 47-58; Levrero, M. et al., J Hepatol (2009), 51 (3), 581-592). The current SoC could not eliminate the cccDNA which are already present in the infected cells.

There is an urgent need to discover and develop new anti-HBV reagents to eliminate or permanently silence cccDNA, the source of chronicity (Ahmed, M. et al., Drug Discov Today (2015), 20 (5), 548-561; Nassal, M., Gut (2015), 64 (12), 1972-1984).

### SUMMARY OF THE INVENTION

Objects of the present invention are novel compounds of formula (I), their manufacture, medicaments based on a compound in accordance with the invention and their production as well as the use of compounds of formula (I) as HBV cccDNA inhibitors and for the treatment or prophylaxis of HBV infection. The compounds of formula (I) show superior anti-HBV activity.

The present invention relates to a compound of formula (I)

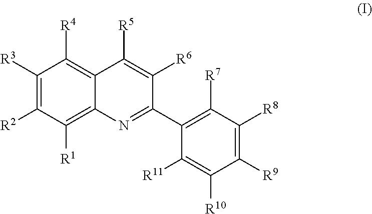

wherein
R¹ is H, halogen, cyano, C₁₋₆alkyl or haloC₁₋₆alkyl;
R² is H, halogen, cyano, C₁₋₆alkyl or haloC₁₋₆alkyl;
R³ is H, halogen, cyano, C₁₋₆alkyl or haloC₁₋₆alkyl;
R⁴ is H, halogen, cyano, C₁₋₆alkyl or haloC₁₋₆alkyl;
R⁵ is H, halogen, haloC₁₋₆alkyl, C₁₋₆alkyl, carboxy, C₁₋₆alkoxy, C₁₋₆alkoxyC₁₋₆alkyl, C₁ alkoxycarbonyl, C₁₋₆alkyl-C(O)O—C₁₋₆alkyl-, hydroxyC₁₋₆alkyl or hydroxy;
R⁶ is H, C₁₋₆alkoxy (carboxyC₃₋₇cycloalkoxy)C₁₋₆alkoxy or (C₁₋₆alkoxycarbonylC₃₋₇cycloalkoxy)C₁₋₆alkoxy;
R⁷ is H, halogen, cyano, C₁₋₆alkyl or haloC₁₋₆alkyl;
R⁸ is H, halogen, cyano, C₁₋₆alkyl or haloC₁₋₆alkyl;
R⁹ is H, hydroxy, C₁₋₆alkoxy, hydroxyC₁₋₆alkoxy, (carboxyC₁₋₆alkoxy)C₁₋₆alkoxy, carboxyphenylC₁₋₆alkoxy, (carboxyC₃₋₇cycloalkoxy)C₁₋₆alkoxy or (C₁₋₆alkoxycarbonylC₃₋₇cycloalkoxy)C₁₋₆alkoxy;
R¹⁰ is H, halogen, cyano, C₁₋₆alkyl or haloC₁₋₆alkyl;
R¹¹ is H, halogen, cyano, C₁₋₆alkyl or haloC₁₋₆alkyl;
wherein with the proviso that R¹ and R² are not H simultaneously; or that R⁸ and R⁹ are not H simultaneously;
or a pharmaceutically acceptable salt thereof.

### DETAILED DESCRIPTION OF THE INVENTION

### Definitions

As used herein, the term “C₁₋₆alkyl” denotes a saturated, linear- or branched chain alkyl group containing 1 to 6, particularly 1 to 4 carbon atoms, for example methyl, ethyl, propyl, isopropyl, 1-butyl, 2-butyl, tert-butyl and the like. Particular “C₁₋₆alkyl” groups are methyl, ethyl, isopropyl and tert-butyl. More particularly, “C₁₋₆alkyl” group is methyl and ethyl.

The term “C₃₋₇cycloalkyl” denotes a saturated carbon ring containing from 3 to 7 carbon atoms, particularly from 3 to 6 carbon atoms, for example, cyclopropyl, cyclobutyl, cyclopentyl, cyclohexyl, cycloheptyl and the like. Particular “C₃₋₇cycloalkyl” group is cyclopropyl, cyclobutyl or cyclopentyl.

The term “C₁₋₆alkoxy” denotes a group C₁₋₆alkyl-O—, wherein the “C₁₋₆alkyl” is as defined above; for example methoxy, ethoxy, propoxy, iso-propoxy, n-butoxy, iso-butoxy, 2-butoxy, tert-butoxy, pentoxy, hexyloxy and the like. Particular “C₁₋₆alkoxy” groups are methoxy, ethoxy and propoxy. More particularly, “C₁₋₆alkoxy” group is methoxy or ethoxy.

The term “C₃₋₇cycloalkoxy” denotes a group C₃₋₇cycloalkyl-O—, wherein the “C₃₋₇cycloalkyl” is as defined above; for example cyclopropoxy, cyclobutoxy, cyclopentoxy.

Particular “C₃₋₇cycloalkoxy” group is cyclobutoxy.

The term “halogen” denotes fluorine, chlorine, bromine or iodine.

The term “carbonyl” denotes the group —C(O)—.

The term “haloC₁₋₆alkyl” denotes an alkyl group wherein at least one of the hydrogen atoms of the alkyl group is replaced by same or different halogen atoms, particularly fluoro atoms. Examples of haloC₁₋₆alkyl include monofluoro-, monochloro-, difluoro- or trifluoromethyl, -ethyl or -propyl, for example chloroethyl, 3,3,3-trifluoropropyl, 2-fluoroethyl, 2,2,2-trifluoroethyl, fluoromethyl, difluoromethyl, trifluoromethyl and trifluoroethyl.

The compounds according to the present invention may exist in the form of their pharmaceutically acceptable salts. The term “pharmaceutically acceptable salt” refers to conventional acid-addition salts or base-addition salts that retain the biological effectiveness and properties of the compounds of formula (I) and are formed from suitable non-toxic organic or inorganic acids or organic or inorganic bases. Acid-addition salts include for example those derived from inorganic acids such as hydrochloric acid, hydrobromic acid, hydroiodic acid, sulfuric acid, sulfamic acid, phosphoric acid and nitric acid, and those derived from organic acids such as p-toluenesulfonic acid, salicylic acid, methanesulfonic acid, oxalic acid, succinic acid, citric acid, malic acid, lactic acid, fumaric acid, and the like. Base-addition salts include those derived from ammonium, potassium, sodium and, quaternary ammonium hydroxides, such as for example, tetramethyl ammonium hydroxide. The chemical modification of a pharmaceutical compound into a salt is a technique well known to pharmaceutical chemists in order to obtain improved physical and chemical stability, hygroscopicity, flowability and solubility of compounds. It is for example described in Bastin R. J., et al., Organic Process Research & Development 2000, 4, 427-435. Particular are the sodium salts of the compounds of formula (I).

cccDNA INHIBITORS

The present invention provides (i) a compound having the general formula (I):

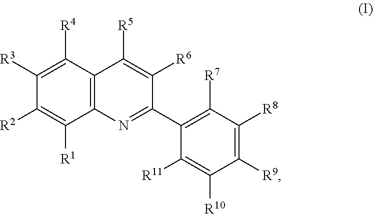

wherein
R¹ is H, halogen, cyano, C₁₋₆alkyl or haloC₁₋₆alkyl;
R² is H, halogen, cyano, C₁₋₆alkyl or haloC₁₋₆alkyl;
R³ is H, halogen, cyano, C₁₋₆alkyl or haloC₁₋₆alkyl;
R⁴ is H, halogen, cyano, C₁₋₆alkyl or haloC₁₋₆alkyl;
R⁵ is H, halogen, haloC₁₋₆alkyl, C₁₋₆alkyl, carboxy, C₁₋₆alkoxy, C₁₋₆alkoxyC₁₋₆alkyl, C₁₋₆alkoxycarbonyl, C₁₋₆alkyl-C(O)O—C₁₋₆alkyl-, hydroxyC₁₋₆alkyl or hydroxy;
R⁶ is H, C₁₋₆alkoxy (carboxyC₃₋₇cycloalkoxy)C₁₋₆alkoxy or (C₁₋₆alkoxycarbonylC₃₋₇cycloalkoxy)C₁₋₆alkoxy;
R⁷ is H, halogen, cyano, C₁₋₆alkyl or haloC₁₋₆alkyl;
R⁸ is H, halogen, cyano, C₁₋₆alkyl or haloC₁₋₆alkyl;
R⁹ is H, hydroxy, C₁₋₆alkoxy, hydroxyC₁₋₆alkoxy, (carboxyC₁₋₆alkoxy)C₁₋₆alkoxy, carboxyphenylC₁₋₆alkoxy, (carboxyC₃₋₇cycloalkoxy)C₁₋₆alkoxy or (C₁₋₆alkoxycarbonylC₃₋₇cycloalkoxy)C₁₋₆alkoxy;
R¹⁰ is H, halogen, cyano, C₁₋₆alkyl or haloC₁₋₆alkyl;
R¹¹ is H, halogen, cyano, C₁₋₆alkyl or haloC₁₋₆alkyl;
wherein with the proviso that R¹ and R² are not H simultaneously; or that R⁸ and R⁹ are not H simultaneously;
or a pharmaceutically acceptable salt thereof.

A further embodiment of the present invention is (i-1) a compound of formula (I), wherein,

wherein
R¹ is H or halogen;
R² is H, halogen or haloC₁₋₆alkyl;
R³ is H or halogen;
R⁴ is H or halogen;
R⁵ is H, halogen, haloC₁₋₆alkyl, C₁₋₆alkyl, carboxy, C₁₋₆alkoxy, C₁₋₆alkoxyC₁₋₆alkyl, C₁₋₆alkoxycarbonyl, C₁₋₆alkyl-C(O)O—C₁₋₆alkyl-, hydroxyC₁₋₆alkyl or hydroxy;
R⁶ is H, C₁₋₆alkoxy or (carboxyC₃₋₇cycloalkoxy)C₁₋₆alkoxy;
R⁷ is H or cyano;
R⁸ is H, halogen or haloC₁₋₆alkyl;
R⁹ is H, hydroxy, C₁₋₆alkoxy, hydroxyC₁₋₆alkoxy, (carboxyC₁₋₆alkoxy)C₁₋₆alkoxy, carboxyphenylC₁₋₆alkoxy, (carboxyC₃₋₇cycloalkoxy)C₁₋₆alkoxy or (C₁₋₆alkoxycarbonylC₃₋₇cycloalkoxy)C₁₋₆alkoxy;
R¹⁰ is H, halogen or haloC₁₋₆alkyl;
R¹¹ is H, halogen or haloC₁₋₆alkyl;
wherein with the proviso that R¹ and R² are not H simultaneously; or that R⁸ and R⁹ are not H simultaneously;
or a pharmaceutically acceptable salt.

A further embodiment of the present invention is (ii) a compound of formula (I), wherein,

R¹ is H or halogen;
R² is H or haloC₁₋₆alkyl;

### R³ is H;

R⁴ is H or halogen;
R⁵ is H, C₁₋₆alkyl, carboxy, C₁₋₆alkoxy or haloC₁₋₆alkyl;
R⁶ is H, C₁₋₆alkoxy or (carboxyC₃₋₇cycloalkoxy)C₁₋₆alkoxy;
R⁷ is H or cyano;
R⁸ is H, halogen or haloC₁₋₆alkyl;
R⁹ is H, hydroxy, C₁₋₆alkoxy, hydroxyC₁₋₆alkoxy, (carboxyC₁₋₆alkoxy)C₁₋₆alkoxy, carboxyphenylC₁₋₆alkoxy, (carboxyC₃₋₇cycloalkoxy)C₁₋₆alkoxy or (C₁₋₆alkoxycarbonylC₃₋₇cycloalkoxy)C₁₋₆alkoxy;

### R¹⁰ is H;

### R¹¹ is H;

wherein with the proviso that R¹ and R² are not H simultaneously; or that R⁸ and R⁹ are not H simultaneously;
or a pharmaceutically acceptable salt thereof.

A further embodiment of the present invention is (iii) a compound of formula (I), wherein,

R¹ is H or chloro;
R² is H or CF₃;

### R³ is H;

R⁴ is H or chloro;
R⁵ is H, methyl, methoxy, carboxy or CF₃;
R⁶ is H, methoxy or (carboxycyclobutoxy)ethoxy;
R⁷ is H or cyano;
R⁸ is H, bromo or CF₃;
R⁹ is H, hydroxy, methoxy, (carboxycyclobutoxy)ethoxy, (carboxymethoxy)ethoxy, hydroxyethoxy, (carboxyphenyl)methoxy or (methoxycarbonylcyclobutoxy)ethoxy;

### R¹⁰ is H;

### R¹¹ is H;

wherein with the proviso that R¹ and R² are not H simultaneously; or that R⁸ and R⁹ are not H simultaneously;
or a pharmaceutically acceptable salt thereof.

A further embodiment of the present invention is (iv) a compound of formula (I) or pharmaceutically acceptable salt thereof, wherein R⁵ is H haloC₁₋₆alkyl.

A further embodiment of the present invention is (v) a compound of formula (I) or pharmaceutically acceptable salt thereof, wherein R⁵ is H or CF₃.

A further embodiment of the present invention is (vi) a compound of formula (I) or pharmaceutically acceptable salt thereof, wherein R⁸ is H or haloC₁₋₆alkyl.

A further embodiment of the present invention is (vii) a compound of formula (I) or pharmaceutically acceptable salt thereof, wherein R⁸ is H or CF₃.

A further embodiment of the present invention is (viii) a compound of formula (I) or pharmaceutically acceptable salt thereof, wherein R⁹ is H, hydroxyC₁₋₆alkoxy, (carboxyC₁₋₆alkoxy)C₁₋₆alkoxy or (carboxyC₃₋₇cycloalkoxy)C₁₋₆alkoxy.

A further embodiment of the present invention is (ix) a compound of formula (I) or pharmaceutically acceptable salt thereof, wherein R⁹ is H, (carboxycyclobutoxy)ethoxy, (carboxymethoxy)ethoxy or hydroxyethoxy.

A further embodiment of the present invention is (x) a compound of formula (I), wherein,

R¹ is halogen;

### R² is H;

### R³ is H;

R⁴ is H or halogen;
R⁵ is H or haloC₁₋₆alkyl;
R⁶ is H, C₁₋₆alkoxy or (carboxyC₃₋₇cycloalkoxy)C₁₋₆alkoxy;

### R⁷ is H;

R⁸ is H or haloC₁₋₆alkyl;
R⁹ is hydroxy, (carboxyC₁₋₆alkoxy)C₁₋₆alkoxy or (carboxyC₃₋₇cycloalkoxy)C₁₋₆alkoxy;

### R¹⁰ is H;

### R¹¹ is H;

or a pharmaceutically acceptable salt thereof.

A further embodiment of the present invention is (xi) a compound of formula (I), wherein,

R¹ is chloro;

### R² is H;

### R³ is H;

R⁴ is H or chloro;
R⁵ is H or CF₃;
R⁶ is H, methoxy or (carboxycyclobutoxy)ethoxy;

### R⁷ is H;

R⁸ is H or CF₃;
R⁹ is hydroxy, (carboxymethoxy)ethoxy or (carboxycyclobutoxy)ethoxy;

### R¹⁰ is H;

### R¹¹ is H;

or a pharmaceutically acceptable salt thereof.

A further embodiment of the present invention is (xii) a compound of formula (I) or pharmaceutically acceptable salt thereof, wherein with the proviso that R¹ and R² are not H simultaneously; and that R⁸ and R⁹ are not H simultaneously.

In another embodiment (xiii) of the present invention, particular compounds of the present invention are selected from:
cis-3-[2-[4-(8-chloro-2-quinolyl)phenoxy]ethoxy]cyclobutanecarboxylic acid;
2-[2-[4-(8-chloro-2-quinolyl)phenoxy]ethoxy]acetic acid;
8-chloro-2-[4-methoxy-3-(trifluoromethyl)phenyl]quinoline;
2-[4-(8-chloro-2-quinolyl)-2-(trifluoromethyl)phenoxy]ethanol;
cis-3-[2-[4-(8-chloro-2-quinolyl)-2-(trifluoromethyl)phenoxy]ethoxy]cyclobutanecarboxylic acid;
2-[2-[4-(8-chloro-2-quinolyl)-2-(trifluoromethyl)phenoxy]ethoxy]acetic acid;
cis-3-[2-[4-(8-chloro-2-quinolyl)-3-cyano-phenoxy]ethoxy]cyclobutanecarboxylic acid;
2-[4-[7-(trifluoromethyl)-2-quinolyl]phenoxy]ethanol;
cis-methyl 3-[2-[4-[7-(trifluoromethyl)-2-quinolyl]phenoxy]ethoxy]cyclobutanecarboxylate;
2-(3-bromophenyl)-7-(trifluoromethyl)quinoline-4-carboxylic acid;
2-(3-bromophenyl)-4-methyl-7-(trifluoromethyl)quinolone;
3-[2-[4-[8-chloro-4-(trifluoromethyl)-2-quinolyl]phenoxy]ethoxy]cyclobutanecarboxylic acid;
2-[2-[4-[8-chloro-4-(trifluoromethyl)-2-quinolyl]phenoxy]ethoxy]acetic acid;
3-[[4-(8-chloro-4-methoxy-2-quinolyl)phenoxy]methyl]benzoic acid;
cis-3-[2-[4-(8-chloro-3-methoxy-2-quinolyl)phenoxy]ethoxy]cyclobutanecarboxylic acid and
3-[2-[[5,8-dichloro-2-(4-hydroxyphenyl)-3-quinolyl]oxy]ethoxy]cyclobutanecarboxylic acid;
   or a pharmaceutically acceptable salt thereof.

In another embodiment (xiv) of the present invention, particular compounds of the present invention are selected from:
cis-3-[2-[4-(8-chloro-2-quinolyl)phenoxy]ethoxy]cyclobutanecarboxylic acid;
2-[2-[4-(8-chloro-2-quinolyl)phenoxy]ethoxy]acetic acid;
cis-3-[2-[4-(8-chloro-2-quinolyl)-2-(trifluoromethyl)phenoxy]ethoxy]cyclobutanecarboxylic acid;
2-[2-[4-(8-chloro-2-quinolyl)-2-(trifluoromethyl)phenoxy]ethoxy]acetic acid;
3-[2-[4-[8-chloro-4-(trifluoromethyl)-2-quinolyl]phenoxy]ethoxy]cyclobutanecarboxylic acid;
2-[2-[4-[8-chloro-4-(trifluoromethyl)-2-quinolyl]phenoxy]ethoxy]acetic acid;
cis-3-[2-[4-(8-chloro-3-methoxy-2-quinolyl)phenoxy]ethoxy]cyclobutanecarboxylic acid and
3-[2-[[5,8-dichloro-2-(4-hydroxyphenyl)-3-quinolyl]oxy]ethoxy]cyclobutanecarboxylic acid;
   or a pharmaceutically acceptable salt thereof.

### Synthesis

The compounds of the present invention can be prepared by any conventional means. Suitable processes for synthesizing these compounds as well as their starting materials are provided in the schemes below and in the subsequent examples. All substituents, in particular, R¹ to R¹², X, G₁ G₂, Q are defined above unless otherwise indicated. Furthermore, and unless explicitly otherwise stated, all reactions, reaction conditions, abbreviations and symbols have the meanings well known to a person of ordinary skill in the art.

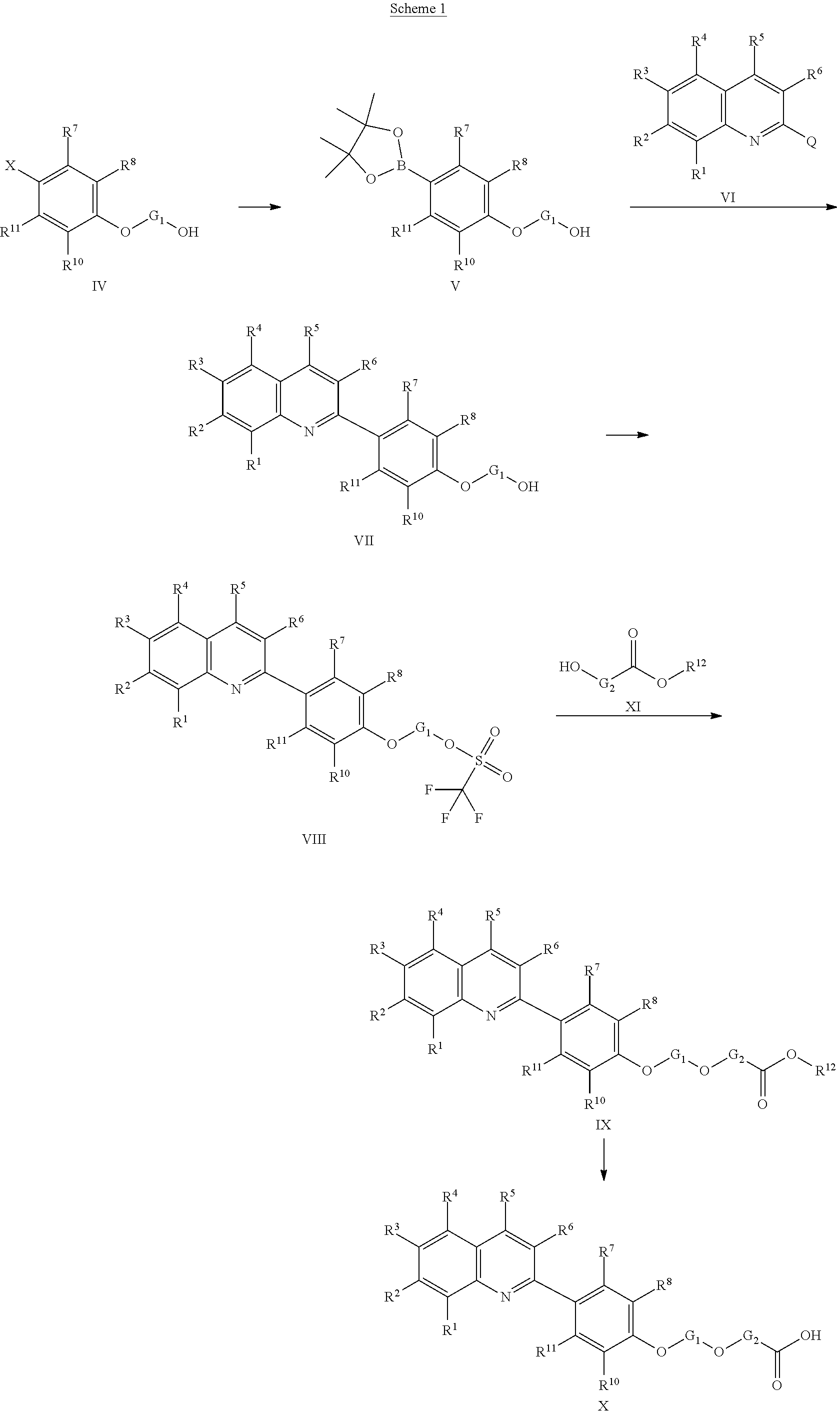

wherein X is halogen; Q is halogen or OTf; R¹² is C₁₋₆alkyl; G₁ is C₁₋₆alkyl; and G₂ is C₁₋₆alkyl or C₃₋₇cycloalkyl.

Treatment of compound of formula IV with bis(pinacolato)diboron in the presence of a suitable base, such as potassium acetate and a suitable palladium catalyst, such as PdCl₂(DPPF)—CH₂Cl₂ adduct, in a suitable solvent, such as DMSO, affords corresponding boronic acid pinacol ester V. Coupling of compound of formula V with 2-halogen substituted or 2-trifluoromethanesulfonate substituted quinoline VI in the presence of a suitable palladium catalyst, such as Pd(PPh₃)₄, in a suitable solvent, such as dioxane or toluene, affords compound of formula VII. Esterification of compound of formula VII with trifluoromethanesulfonic anhydride in the presence of a suitable base, such as 2,6-lutidine, in a suitable solvent, such as DCM, affords trifluoromethanesulfonate VIII. Substitution of trifluoromethanesulfonate VIII with compound of formula XI in the presence of a suitable base, such as NaH, in a suitable solvent, such as DMSO, affords compound of formula IX. Hydrolysis of compound of formula IX in the presence of suitable base, such as LiOH, affords corresponding acid X.

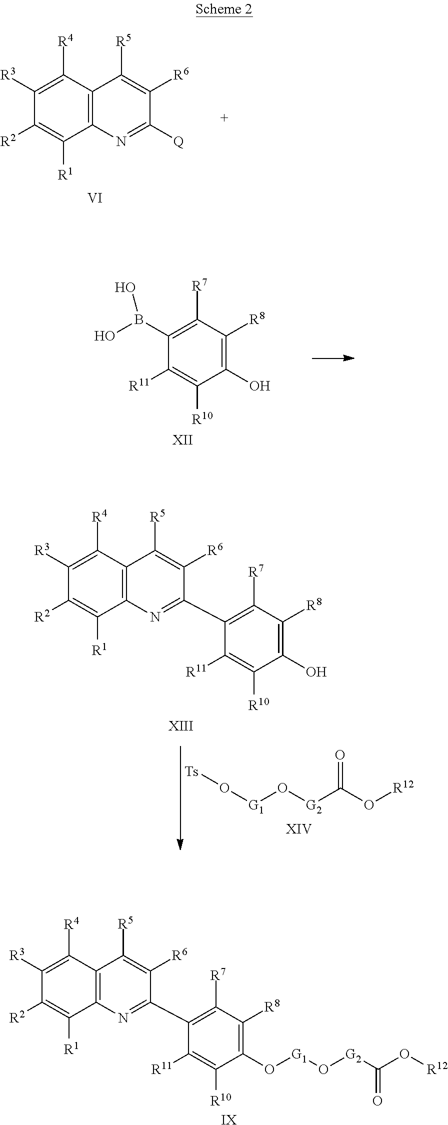

wherein Q is halogen or OTf; R¹² is C₁₋₆alkyl; G₁ is C₁₋₆alkyl; and G₂ is C₁₋₆alkyl or C₃₋₇cycloalkyl.

Coupling of 2-halogen substituted or 2-trifluoromethanesulfonate substituted quinoline VI and boronic acid XII in the presence of a suitable palladium catalyst, such as Pd(PPh₃)₄, in a suitable solvent, such as dioxane or toluene, affords compound of formula XIII. Substitution of compound of formula XIII with compound of formula XIV in the presence of a suitable base such as K₂CO₃, in a suitable solvent such as DMF, also affords compound of formula IX.

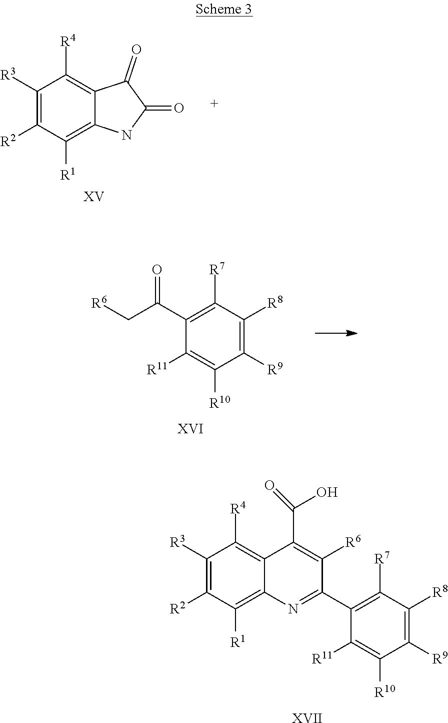

Condensation of compound of formula XV with compound of formula XVI in the presence of suitable base, such as KOH, in a suitable solvent, such as ethanol, affords compound of formula XVII.

This invention also relates to a process for the preparation of a compound of formula (I) comprising any one of the following steps:

(a) Hydrolysis of compound of formula (IX),

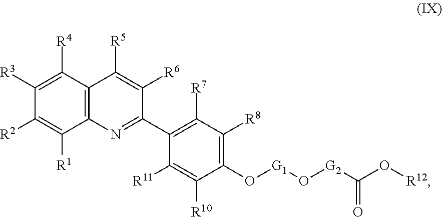

in the presence of a base;
(b) Suzuki crossing coupling reaction of quinoline (VI),

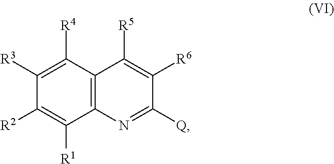

with borate ester (V) in the presence of a catalyst;
(c) Substitution of trifluoromethanesulfonate(VIII),

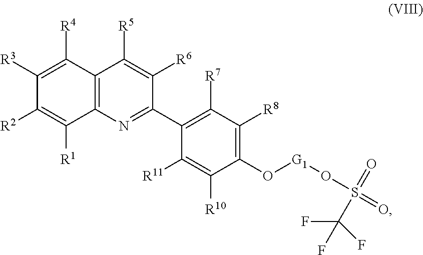

with the compound of formula (XI) in the presence of a base;
(d) Suzuki crossing coupling reaction of quinoline (VI) with boronic acid (XII) in the presence of a catalyst;
(e) Substitution of trifluoromethanesulfonate(XIII),

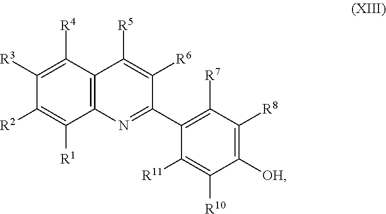

with the compound of formula (XIV) in the presence of a base;
(f) Condensation of compound of formula (XV),

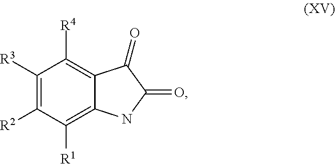

with the compound of formula (XVI) in the presence of a base;
wherein R¹, R², R³, R⁴, R⁵, R⁶, R⁷, R⁸, R⁹, R¹⁰ and R¹¹ are defined as above; R¹² is C₁₋₆alkyl; G₁ is C₁₋₆alkyl; G₂ is C₁₋₆alkyl or C₃₋₇cycloalkyl; Q is halogen or OTf.
the base in step (a), can be for example NaH;
the catalyst in step (b) can be for example Pd(PPh₃)₄;
the base in step (c), can be for example NaH;
the catalyst in step (d), can be for example Pd(PPh₃)₄;
the base in step (e), can be for example K₂CO₃;
the base in step (f), can be for example KOH.

A compound of formula (I) when manufactured according to the above process is also an object of the invention.

### Pharmaceutical Compositions and Administration

The invention also relates to a compound of formula (I) for use as therapeutically active substance. Another embodiment provides pharmaceutical compositions or medicaments containing the compounds of the invention and a therapeutically inert carrier, diluent or excipient, as well as methods of using the compounds of the invention to prepare such compositions and medicaments. In one example, compounds of formula (I) may be formulated by mixing at ambient temperature at the appropriate pH, and at the desired degree of purity, with physiologically acceptable carriers, i.e., carriers that are non-toxic to recipients at the dosages and concentrations employed into a galenical administration form. The pH of the formulation depends mainly on the particular use and the concentration of compound, but preferably ranges anywhere from about 3 to about 8. In one example, a compound of formula (I) is formulated in an acetate buffer, at pH 5. In another embodiment, the compounds of formula (I) are sterile. The compound may be stored, for example, as a solid or amorphous composition, as a lyophilized formulation or as an aqueous solution.

Compositions are formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The “effective amount” of the compound to be administered will be governed by such considerations, and is the minimum amount necessary to inhibit cccDNA in HBV patients, consequently lead to the reduction of HBsAg and HBeAg (HBV e antigen) in serum. For example, such amount may be below the amount that is toxic to normal cells, or the mammal as a whole.

In one example, the pharmaceutically effective amount of the compound of the invention administered parenterally per dose will be in the range of about 0.1 to 100 mg/kg, alternatively about 0.1 to 50 mg/kg of patient body weight per day, with the typical initial range of compound used being 0.3 to 15 mg/kg/day. In another embodiment, oral unit dosage forms, such as tablets and capsules, preferably contain from about 25 to about 1000 mg of the compound of the invention.

The compounds of the invention may be administered by any suitable means, including oral, topical (including buccal and sublingual), rectal, vaginal, transdermal, parenteral, subcutaneous, intraperitoneal, intrapulmonary, intradermal, intrathecal and epidural and intranasal, and, if desired for local treatment, intralesional administration. Parenteral infusions include intramuscular, intravenous, intraarterial, intraperitoneal, or subcutaneous administration.

The compounds of the present invention may be administered in any convenient administrative form, e.g., tablets, powders, capsules, solutions, dispersions, suspensions, syrups, sprays, suppositories, gels, emulsions, patches, etc. Such compositions may contain components conventional in pharmaceutical preparations, e.g., diluents, carriers, pH modifiers, sweeteners, bulking agents, and further active agents.

A typical formulation is prepared by mixing a compound of the present invention and a carrier or excipient. Suitable carriers and excipients are well known to those skilled in the art and are described in detail in, e.g., Ansel, Howard C., et al., Ansel's Pharmaceutical Dosage Forms and Drug Delivery Systems. Philadelphia: Lippincott, Williams & Wilkins, 2004; Gennaro, Alfonso R., et al. Remington: The Science and Practice of Pharmacy. Philadelphia: Lippincott, Williams & Wilkins, 2000; and Rowe, Raymond C. Handbook of Pharmaceutical Excipients. Chicago, Pharmaceutical Press, 2005. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents, diluents and other known additives to provide an elegant presentation of the drug (i.e., a compound of the present invention or pharmaceutical composition thereof) or aid in the manufacturing of the pharmaceutical product (i.e., medicament).

An example of a suitable oral dosage form is a tablet containing about 25 to 500 mg of the compound of the invention compounded with about 90 to 30 mg anhydrous lactose, about 5 to 40 mg sodium croscarmellose, about 5 to 30 mg polyvinylpyrrolidone (PVP) K30, and about 1 to 10 mg magnesium stearate. The powdered ingredients are first mixed together and then mixed with a solution of the PVP. The resulting composition can be dried, granulated, mixed with the magnesium stearate and compressed to tablet form using conventional equipment. An example of an aerosol formulation can be prepared by dissolving the compound, for example 5 to 400 mg, of the invention in a suitable buffer solution, e.g. a phosphate buffer, adding a tonicifier, e.g. a salt such sodium chloride, if desired. The solution may be filtered, e.g., using a 0.2 micron filter, to remove impurities and contaminants.

An embodiment, therefore, includes a pharmaceutical composition comprising a compound of formula (I), or a pharmaceutically acceptable salt thereof.

In a further embodiment includes a pharmaceutical composition comprising a compound of formula (I), or a pharmaceutically acceptable salt thereof, together with a pharmaceutically acceptable carrier or excipient.

Another embodiment includes a pharmaceutical composition comprising a compound of formula (I), or a pharmaceutically acceptable salt thereof for use in the treatment of HBV infection.

### Indications and Methods of Treatment

The compounds of the invention can inhibit cccDNA and have anti-HBV activity. Accordingly, the compounds of the invention are useful for the treatment or prophylaxis of HBV infection.

The invention relates to the use of a compound of formula (I) for the inhibition of cccDNA.

The invention also relates to the use of a compound of formula (I) for the inhibition of HBeAg.

The invention further relates to the use of a compound of formula (I) for the inhibition of HBsAg.

The invention relates to the use of a compound of formula (I) for the inhibition of HBV DNA.

The invention relates to the use of a compound of formula (I) for the treatment or prophylaxis of HBV infection.

The use of a compound of formula (I) for the preparation of medicaments useful in the treatment or prophylaxis diseases that are related to HBV infection is an object of the invention.

The invention relates in particular to the use of a compound of formula (I) for the preparation of a medicament for the treatment or prophylaxis of HBV infection.

Another embodiment includes a method for the treatment or prophylaxis of HBV infection, which method comprises administering an effective amount of a compound of Formula (I), or a pharmaceutically acceptable salt thereof.

The invention also relates to a method for the inhibition of cccDNA, which method comprises administering an effective amount of a compound of Formula (I), or a pharmaceutically acceptable salt thereof.

The invention also relates to a method for the inhibition of HbeAg, which method comprises administering an effective amount of a compound of Formula (I), or a pharmaceutically acceptable salt thereof.

The invention also relates to a method for the inhibition of HBsAg, which method comprises administering an effective amount of a compound of Formula (I), or a pharmaceutically acceptable salt thereof.

The invention also relates to a method for the inhibition of HBV DNA, which method comprises administering an effective amount of a compound of Formula (I), or a pharmaceutically acceptable salt thereof.

### Examples

The invention will be more fully understood by reference to the following examples. They should not, however, be construed as limiting the scope of the invention.

### Abbreviations Used Herein are as Follows:

PE: petroleum ether
BBr₃: boron tribromide
DMAP: 4-dimethylaminopyridine

### DMF: N,N-dimethylformamide

### DPPF: 1,1′-Bis(diphenylphosphino)ferrocene

EC₅₀: the molar concentration of an inhibitor, which produces 50% of the maximum
possible response for that inhibitor.
FBS: fetal bovine serum
HPLC: high performance liquid chromatography
hr(s): hour(s)
min: minute
MS (ESI): mass spectroscopy (electron spray ionization)
Ms: methylsulfonyl
obsd: observed
PE: petroleum ether
PPh₃: triphenylphosphine
rt: room temperature
Tf: trifluoromethanesulfonyl
TFA: trifluoroacetic acid
THF: tetrahydrofuran
TEA: trimethylamine
TMS: trimethylsilyl
LiAlH₄: lithium aluminium hydride
δ: chemical shift

### General Experimental Conditions

Intermediates and final compounds were purified by flash chromatography using one of the following instruments: i) Biotage SP1 system and the Quad 12/25 Cartridge module. ii) ISCO combi-flash chromatography instrument. Silica gel Brand and pore size: i) KP-SIL 60 Å, particle size: 40-60 μm; ii) CAS registry NO: Silica Gel: 63231-67-4, particle size: 47-60 micron silica gel; iii) ZCX from Qingdao Haiyang Chemical Co., Ltd, pore: 200-300 or 300-400.

Intermediates and final compounds were purified by preparative HPLC on reversed phase column using X Bridge™ Perp C₁₈ (5 μm, OBD™ 30×100 mm) column or SunFire™ Perp C₁₈ (5 μm, OBD™ 30×100 mm) column.

LC/MS spectra were obtained using a Waters UPLC-SQD Mass. Standard LC/MS conditions were as follows (running time 3 minutes):

Acidic condition: A: 0.1% formic acid and 1% acetonitrile in H₂O; B: 0.1% formic acid in acetonitrile;

Basic condition: A: 0.05% NH₃.H₂O in H₂O; B: acetonitrile.

Mass spectra (MS): generally only ions which indicate the parent mass are reported, and unless otherwise stated the mass ion quoted is the positive mass ion (M+H)*.

NMR Spectra were obtained using Bruker Avance 400 MHz.

All reactions involving air-sensitive reagents were performed under an argon atmosphere. Reagents were used as received from commercial suppliers without further purification unless otherwise noted.

### PREPARATIVE EXAMPLES

### Example 1

### Cis-3-[2-[4-(8-chloro-2-quinolyl)phenoxy]ethoxy]cyclobutanecarboxylic acid

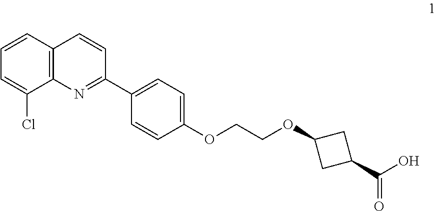 [SMILES: O=C(O)[C@H]1C[C@@H](OCCOc2ccc(-c3ccc4cccc(Cl)c4n3)cc2)C1]

### Step 1: Preparation of 2-[4-(4,4,5,5-tetramethyl-1,3,2-dioxaborolan-2-yl)phenoxy]ethanol

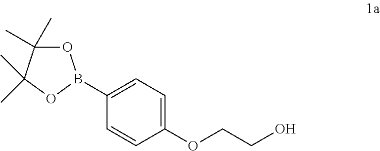 [SMILES: CC1(C)OB(c2ccc(OCCO)cc2)OC1(C)C]

A mixture of 2-(4-bromophenoxy) ethanol (CAS #: 24743-88-9, Cat. #: SY02949, from Accela ChemBio Inc, 4.34 g, 20 mmol), bis(pinacolato)diboron (5.59 g, 22 mmol), PdCl₂(DPPF)—CH₂Cl₂ adduct (439 mg, 600 μmol) and potassium acetate (5.89 g, 60 mmol) in DMSO (50 mL) was stirred at 90° C. under nitrogen atmosphere for 4 hours. The mixture was then diluted with EtOAc (300 mL) and washed with water (100 mL) twice, brine (100 mL) in sequence. The organic layer was separated out and concentrated in vacuo, the residue was purified by column chromatography on silica gel (elution with PE: EtOAc 10:1˜2:1) to give 2-[4-(4,4,5,5-tetramethyl-1,3,2-dioxaborolan-2-yl)phenoxy]ethanol (4.3 g, 81.4% yield) as a light yellow oil. MS obsd. (ESI⁺) [(M+H)⁺]:265.1.

### Step 2: Preparation of (8-chloro-2-quinolyl) Trifluoromethanesulfonate

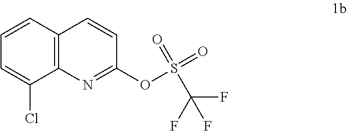 [SMILES: O=S(=O)(Oc1ccc2cccc(Cl)c2n1)C(F)(F)F]

To a solution of 8-chloroquinolin-2-ol (CAS #: 23981-25-1, Cat. #: B162057, from Bepharm, 1.3 g, 7.24 mmol) and 2,6-lutidine (1.01 g, 1.1 mL, 9.41 mmol) in DCM (20 mL) was added trifluoromethanesulfonic anhydride (2.65 g, 1.59 mL, 9.41 mmol) dropwise at 0° C. and the mixture was then stirred at 0° C. for 1 hour. After the reaction was completed, the mixture was diluted with DCM (30 mL) and washed with water (20 mL), 0.5 N HCl (20 mL) and water (20 mL) in sequence. The organic layer was then dried over anhydrous Na₂SO₄ and concentrated in vacuo, the residue was purified by column chromatography on silica gel (elution with PE: EtOAc=10:1˜2:1) to give (8-chloro-2-quinolyl) trifluoromethanesulfonate (2.01 g, 89.1% yield) as a white solid. MS obsd. (ESI⁺) [(M+H)⁺]:312.3.

### Step 3: Preparation of 2-[4-(8-chloro-2-quinolyl)phenoxy]ethanol

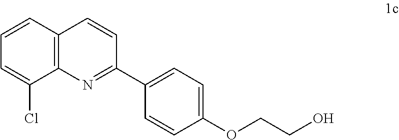 [SMILES: OCCOc1ccc(-c2ccc3cccc(Cl)c3n2)cc1]

A mixture of (8-chloro-2-quinolyl) trifluoromethanesulfonate (500 mg, 1.6 mmol), 2-[4-(4,4,5,5-tetramethyl-1,3,2-dioxaborolan-2-yl)phenoxy]ethanol (424 mg, 1.6 mmol), Pd(Ph₃P)₄ (55.6 mg, 48.1 μmol) and Cs₂CO₃ (627 mg, 1.93 mmol) in toluene (5 mL) and water (1 mL) was charged with N₂ and stirred at 100° C. under microwave condition for 3 hours. After the reaction was completed, the mixture was diluted with water (10 mL) and extracted with EtOAc (25 mL) three times. The combined organic layer was washed with brine, dried over anhydrous Na₂SO₄ and concentrated in vacuo. The residue was purified by column chromatography on silica gel (elution with PE: EtOAc 10:1˜2:1) to give 2-[4-(8-chloro-2-quinolyl)phenoxy]ethanol (202 mg, 42%) as a light yellow solid. MS obsd. (ESI⁺) [(M+H)⁺]:299.7

### Step 4: Preparation of 2-[4-(8-chloro-2-quinolyl)phenoxy]ethyl trifluoromethanesulfonate

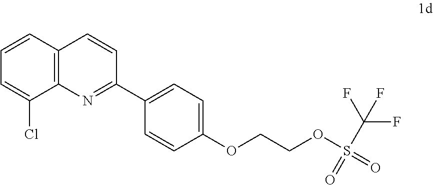 [SMILES: O=S(=O)(OCCOc1ccc(-c2ccc3cccc(Cl)c3n2)cc1)C(F)(F)F]

To a solution of 2-[4-(8-chloro-2-quinolyl)phenoxy]ethanol (90 mg, 300 μmol) and 2,6-lutidine (38.6 mg, 42 μL, 360 μmol) in DCM (5 mL) was added trifluoromethanesulfonic anhydride (102 mg, 60.9 μL, 360 μmol) dropwise at 0° C. and the mixture was stirred at 0° C. for additional 30 minutes. After the reaction was completed, the mixture was diluted with EtOAc (30 mL) and washed with water (10 mL), 0.5 N HCl (10 mL), water (10 mL) and brine (10 mL) in sequence. The organic layer was separated out, dried over anhydrous Na₂SO₄ and concentrated in vacuo to give the crude of 2-[4-(8-chloro-2-quinolyl)phenoxy]ethyl trifluoromethanesulfonate (129 mg, 100% yield) as a yellow oil, which was used in the next step directly without further purification. MS obsd. (ESI⁺) [(M+H)⁺]:432.1.

### Step 5: Preparation of cis-methyl 3-[2-[4-(8-chloro-2-quinolyl)phenoxy]ethoxy]cyclobutanecarboxylate

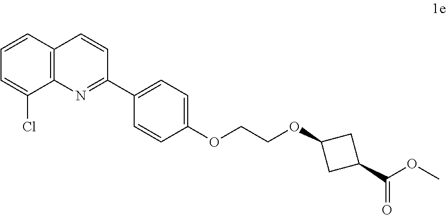 [SMILES: COC(=O)[C@H]1C[C@@H](OCCOc2ccc(-c3ccc4cccc(Cl)c4n3)cc2)C1]

To a mixture of cis-methyl 3-hydroxycyclobutanecarboxylate (CAS #: 63485-50-7, Cat. #: B267690, from Bepharm, 58.6 mg, 450 μmol) and NaH (7.21 mg, 300 μmol) in THF (5 mL) cooled at 0° C. was added 2-[4-(8-chloro-2-quinolyl)phenoxy]ethyl trifluoromethanesulfonate (129 mg, 300 μmol). The mixture was then stirred at 0° C. for 1 hour. After the reaction was completed, the reaction was quenched with water (20 mL). The resulting mixture was extracted with EtOAc (30 mL) twice. The combined organic layer was washed with brine (10 mL), dried over anhydrous Na₂SO₄ and concentrated in vacuo. The residue was purified by column chromatography on silica gel (elution with PE: EtOAc 10:1˜2:1) to give cis-methyl 3-[2-[4-(8-chloro-2-quinolyl)phenoxy]ethoxy]cyclobutanecarboxylate (75 mg, 60.6% over 2 steps) as a light yellow solid. MS obsd. (ESI⁺) [(M+H)⁺]:411.2.

### Step 6: Preparation of cis-3-[2-[4-(8-chloro-2-quinolyl)phenoxy]ethoxy]cyclobutanecarboxylic Acid

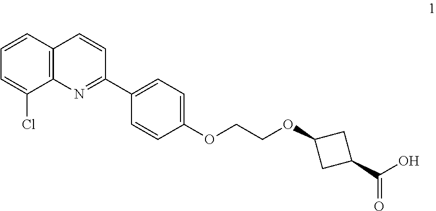 [SMILES: O=C(O)[C@H]1C[C@@H](OCCOc2ccc(-c3ccc4cccc(Cl)c4n3)cc2)C1]

To a solution of cis-methyl 3-[2-[4-(8-chloro-2-quinolyl)phenoxy]ethoxy]cyclobutanecarboxylate (60 mg, 146 μmol) in the mixed solvent of THF (2 mL) and water (2 mL) was added LiOH (18.4 mg, 437 μmol) and the mixture was stirred at room temperature for 3 hours. After the reaction was completed, the mixture was adjusted to pH ˜5 by addition of 1N HCl and the resulting solution was extracted with EtOAc (20 mL) three times. The combined organic layer was dried over anhydrous Na₂SO₄ and concentrated in vacuo to give cis-3-[2-[4-(8-chloro-2-quinolyl)phenoxy]ethoxy]cyclobutanecarboxylic acid (37 mg, 62.6% yield) as a light yellow solid. ¹H NMR (DMSO-d₆, 400 MHz): δ ppm 8.06-8.13 (m, 3H), 7.82 (d, J=8.68 Hz, 1H), 7.69 (dd, J=7.58, 1.22 Hz, 1H), 7.64 (dd, J=8.19, 1.10 Hz, 1H), 7.29 (t, J=7.82 Hz, 1H), 6.86-6.94 (m, 2H), 3.95-4.00 (m, 2H), 3.81-3.93 (m, 1H), 3.59 (dd, J=5.38, 3.91 Hz, 2H), 2.49-2.60 (m, 1H), 2.36-2.46 (m, 2H), 2.00-2.12 (m, 2H). MS obsd. (ESI⁺) [(M+H)⁺]:398.3.

### Example 2

### 2-[2-[4-(8-chloro-2-quinolyl)phenoxy]ethoxy]acetic acid

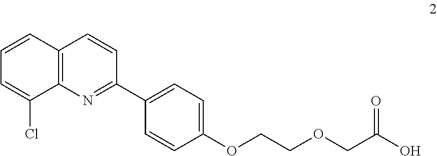 [SMILES: O=C(O)COCCOc1ccc(-c2ccc3cccc(Cl)c3n2)cc1]

Example 2 was prepared in analogy to the procedure described for the preparation of Example 1 by using methyl 2-hydroxyacetate instead of cis-methyl 3-hydroxycyclobutanecarboxylate in Step 5. ¹H NMR (DMSO-d₆, 400 MHz): δ ppm 12.68 (br s, 1 H), 8.49 (d, J=8.68 Hz, 1H), 8.34 (d, J=8.80 Hz, 2H), 8.24 (d, J=8.68 Hz, 1H), 7.91-8.00 (m, 2H), 7.54 (t, J=7.76 Hz, 1H), 7.15 (d, J=8.93 Hz, 2H), 4.23 (dd, J=5.38, 3.67 Hz, 2H), 4.13 (s, 2H), 3.84-3.90 (m, 2H). MS obsd. (ESI⁺) [(M+H)⁺]:358.1.

### Example 3

### 8-chloro-2-[4-methoxy-3-(trifluoromethyl)phenyl]quinoline

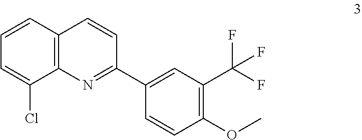 [SMILES: COc1ccc(-c2ccc3cccc(Cl)c3n2)cc1C(F)(F)F]

### Step 1: Preparation of 2-[4-methoxy-3-(trifluoromethyl)phenyl]-4,4,5,5-tetramethyl-1,3,2-dioxaborolane

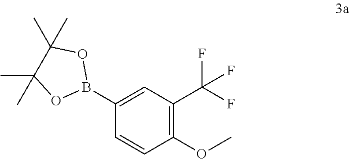 [SMILES: COc1ccc(B2OC(C)(C)C(C)(C)O2)cc1C(F)(F)F]

A mixture of 4-bromo-1-methoxy-2-(trifluoromethyl)benzene (0.5 g, 1.96 mmol), bis(pinacolato) diboron (548 mg, 2.16 mmol), potassium acetate (577 mg, 5.88 mmol,) and PdCl₂(DPPF)—CH₂Cl₂ adduct (28.7 mg, 39.2 μmol) in dioxane (15 mL) was charged with nitrogen and heated at 100° C. for 1 hour. After the reaction was completed, the mixture was then diluted with EtOAc (300 mL) and washed with water (100 mL) twice, brine (100 mL) in sequence. The organic layer was separated out and concentrated in vacuo, the residue was purified by column chromatography on silica gel (elution with PE: EtOAc 10:1˜2:1) to give 2-[4-methoxy-3-(trifluoromethyl)phenyl]-4,4,5,5-tetramethyl-1,3,2-dioxaborolane (470 mg, 79.4% yield) as a light yellow oil. MS obsd. (ESI⁺) [(M+H)⁺]:302.1.

### Step 2: Preparation of 8-chloro-2-[4-methoxy-3-(trifluoromethyl)phenyl]quinoline

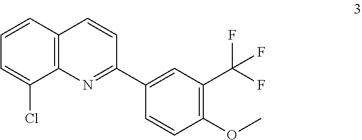 [SMILES: COc1ccc(-c2ccc3cccc(Cl)c3n2)cc1C(F)(F)F]

A mixture of 8-chloroquinolin-2-yl trifluoromethanesulfonate (500 mg, 1.6 mmol), 2-[4-methoxy-3-(trifluoromethyl)phenyl]-4,4,5,5-tetramethyl-1,3,2-dioxaborolane (485 mg, 1.6 mmol), Pd(Ph₃P)₄ (55.6 mg, 48.1 μmol) and Cs₂CO₃ (627 mg, 1.93 mmol) in the mixed solvent of toluene (15 mL) and water (3 mL) was charged with N₂ and the mixture was then stirred at 100° C. under microwave condition for 3 hours. After the reaction was completed, the mixture was diluted with water (10 mL) and extracted with EtOAc (25 mL) three times. The combined organic layer was washed with brine, dried over anhydrous Na₂SO₄ and concentrated in vacuo.

The residue was purified by column chromatography on silica gel (elution with PE: EtOAc 10:1˜2:1) to give 8-chloro-2-[4-methoxy-3-(trifluoromethyl)phenyl]quinoline (400 mg, 72.4%) as a light yellow solid. ¹H NMR (DMSO-d₆, 400 MHz): δ ppm 8.62-8.68 (m, 2H), 8.56 (d, J=8.80 Hz, 1H), 8.36 (d, J=8.68 Hz, 1H), 7.99 (ddd, J=9.66, 8.07, 1.22 Hz, 2H), 7.58 (t, J=7.82 Hz, 1H), 7.49 (d, J=8.56 Hz, 1H), 4.01 (s, 3H). MS obsd. (ESI⁺) [(M+H)⁺]: 338.1.

### Example 4

### 2-[4-(8-chloro-2-quinolyl)-2-(trifluoromethyl)phenoxy]ethanol

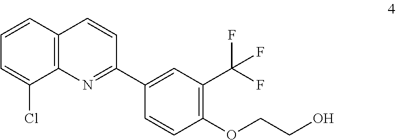 [SMILES: OCCOc1ccc(-c2ccc3cccc(Cl)c3n2)cc1C(F)(F)F]

### Step 1: Preparation of 4-(8-chloro-2-quinolyl)-2-(trifluoromethyl)phenol

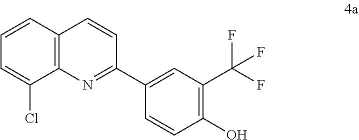 [SMILES: Oc1ccc(-c2ccc3cccc(Cl)c3n2)cc1C(F)(F)F]

To a solution of 8-chloro-2-(4-methoxy-3-(trifluoromethyl)phenyl)quinoline (350 mg, 1.04 mmol) in DCM (10 mL) was added BBr₃ solution (1 mol/L in DCM, 5 mL, 5 mmol) and the mixture was stirred at room temperature overnight. After the reaction was completed, the mixture was quenched by ice water carefully to give a yellow suspension. The mixture was filtered, the solid was collected and dried in vacuo to give the crude product of 4-(8-chloro-2-quinolyl)-2-(trifluoromethyl)phenol (310 mg, 92.4%) as a yellow solid, which was used in the next step directly without further purification. MS obsd. (ESI⁺) [(M+H)⁺]:323.7.

### Step 2: Preparation of methyl 2-[4-(8-chloro-2-quinolyl)-2-(trifluoromethyl)phenoxy]acetate

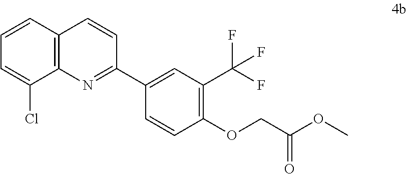 [SMILES: COC(=O)COc1ccc(-c2ccc3cccc(Cl)c3n2)cc1C(F)(F)F]

A mixture of 4-(8-chloro-2-quinolyl)-2-(trifluoromethyl)phenol (310 mg, 958 μmol), methyl 2-bromoacetate (220 mg, 1.44 mmol) and potassium carbonate (132 mg, 958 μmol) in DMF (5 mL) was stirred at 80° C. for 2 hours. After the reaction was completed, the mixture was diluted with water (10 mL) and extracted with EtOAc (25 mL) three times. The combined organic layer was washed with brine, dried over anhydrous Na₂SO₄ and concentrated in vacuo. The residue was purified by column chromatography on silica gel (elution with PE: EtOAc 10:1˜2:1) to give methyl 2-[4-(8-chloro-2-quinolyl)-2-(trifluoromethyl)phenoxy]acetate (240 mg, 63.3%) as a light yellow solid. MS obsd. (ESI⁺) [(M+H)⁺]:395.7.

### Step 3: Preparation of 2-[4-(8-chloro-2-quinolyl)-2-(trifluoromethyl)phenoxy]ethanol

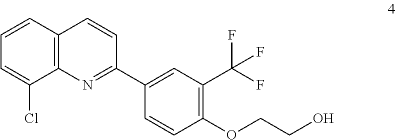 [SMILES: OCCOc1ccc(-c2ccc3cccc(Cl)c3n2)cc1C(F)(F)F]

To a solution of methyl 2-[4-(8-chloro-2-quinolyl)-2-(trifluoromethyl)phenoxy]acetate (240 mg, 606 μmol,) in THF (10 mL) was added LiAlH₄ (69 mg, 1.82 mmol) at 0° C. After completion of addition, the mixture was warmed to room temperature and stirred at room temperature for 2 hours. After the reaction was completed, to the mixture was added sodium sulfate decahydrate (2 g) and the resulting suspension was filtered. The filtrate was dried over anhydrous Na₂SO₄ and concentrated in vacuo to give 2-[4-(8-chloro-2-quinolyl)-2-(trifluoromethyl)phenoxy]ethanol (160 mg, 70.3%) as a light yellow solid. ¹H NMR (METHANOL-d₄, 400 MHz): δ ppm 8.65 (d, J=2.20 Hz, 1H), 8.54 (dd, J=8.74, 2.26 Hz, 1H), 8.41 (d, J=8.68 Hz, 1H), 8.14 (d, J=8.68 Hz, 1H), 7.86-7.92 (m, 2H), 7.48-7.55 (m, 1H), 7.39 (d, J=8.80 Hz, 1H), 4.29 (t, J=5.01 Hz, 2H), 3.94-4.01 (m, 2H). MS obsd. (ESI⁺) [(M+H)⁺]: 368.1.

### Example 5

### Cis-3-[2-[4-(8-chloro-2-quinolyl)-2-(trifluoromethyl)phenoxy]ethoxy]cyclobutanecarboxylic acid

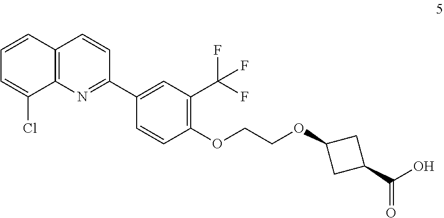 [SMILES: O=C(O)[C@H]1C[C@@H](OCCOc2ccc(-c3ccc4cccc(Cl)c4n3)cc2C(F)(F)F)C1]

### Step 1: Preparation of 2-[4-(8-chloro-2-quinolyl)-2-(trifluoromethyl)phenoxy]ethyl trifluoromethanesulfonate

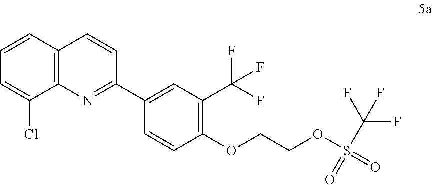 [SMILES: O=S(=O)(OCCOc1ccc(-c2ccc3cccc(Cl)c3n2)cc1C(F)(F)F)C(F)(F)F]

Compound 5a was prepared in analogy to the procedure described for the preparation of Compound id by using 2-[4-(8-chloro-2-quinolyl)-2-(trifluoromethyl)phenoxy]ethanol instead of 2-(4-(8-chloroquinolin-2-yl)phenoxy)ethanol in Step 4.

### Step 2: Preparation of cis-3-[2-[4-(8-chloro-2-quinolyl)-2-(trifluoromethyl)phenoxy]ethoxy]cyclobutanecarboxylic Acid

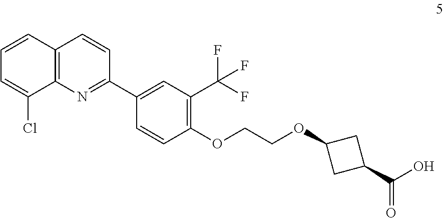 [SMILES: O=C(O)[C@H]1C[C@@H](OCCOc2ccc(-c3ccc4cccc(Cl)c4n3)cc2C(F)(F)F)C1]

Example 5 was prepared in analogy to the procedure described for the preparation of Example 1 by using 2-[4-(8-chloro-2-quinolyl)-2-(trifluoromethyl)phenoxy]ethyl trifluoromethanesulfonate instead of 2-[4-(8-chloro-2-quinolyl)phenoxy]ethyl trifluoromethanesulfonate in Step 5. ¹H NMR (DMSO-d₆, 400 MHz): δ ppm 8.65 (d, J=1.96 Hz, 1H), 8.61 (dd, J=8.80, 2.08 Hz, 1H), 8.55 (d, J=8.80 Hz, 1H), 8.35 (d, J=8.68 Hz, 1H), 7.94-8.03 (m, 2H), 7.57 (t, J=7.83 Hz, 1H), 7.48 (d, J=8.80 Hz, 1H), 4.30-4.35 (m, 2H), 3.84-3.93 (m, 1H), 3.65-3.71 (m, 2H), 2.27-2.39 (m, 3H), 1.90-2.01 (m, 2H). MS obsd. (ESI⁺) [(M+H)⁺]: 466. 1.

### Example 6

### 2-[2-[4-(8-chloro-2-quinolyl)-2-(trifluoromethyl)phenoxy]ethoxy]acetic acid

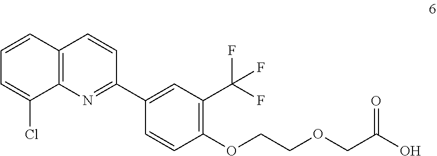 [SMILES: O=C(O)COCCOc1ccc(-c2ccc3cccc(Cl)c3n2)cc1C(F)(F)F]

Example 6 was prepared in analogy to the procedure described for the preparation of Example 1 by using 2-[4-(8-chloro-2-quinolyl)-2-(trifluoromethyl)phenoxy]ethyl trifluoromethanesulfonate and methyl 2-hydroxyacetate instead of 2-[4-(8-chloro-2-quinolyl)phenoxy]ethyl trifluoromethanesulfonate and cis-methyl 3-hydroxycyclobutanecarboxylate in Step 5. ¹H NMR (DMSO-d₆, 400 MHz): δ ppm 12.66 (br s, 1 H), 8.65 (d, J=1.96 Hz, 1H), 8.62 (dd, J=8.80, 2.08 Hz, 1H), 8.56 (d, J=8.80 Hz, 1H), 8.36 (d, J=8.68 Hz, 1H), 7.99 (ddd, J=9.63, 8.10, 1.22 Hz, 2H), 7.58 (t, J=7.82 Hz, 1H), 7.50 (d, J=8.93 Hz, 1H), 4.35-4.42 (m, 2H), 4.15 (s, 2H), 3.85-3.93 (m, 2H). MS obsd. (ESI⁺) [(M+H)⁺]: 426.1

### Example 7

### Cis-3-[2-[4-(8-chloro-2-quinolyl)-3-cyano-phenoxy]ethoxy]cyclobutanecarboxylic acid

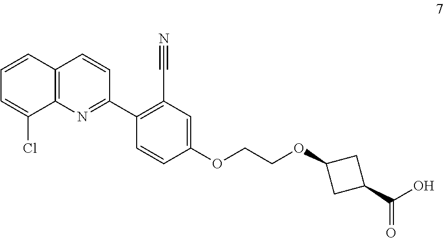 [SMILES: N#Cc1cc(OCCO[C@H]2C[C@@H](C(=O)O)C2)ccc1-c1ccc2cccc(Cl)c2n1]

### Step 1: Preparation of 2-bromo-5-(2-hydroxyethoxy)benzonitrile

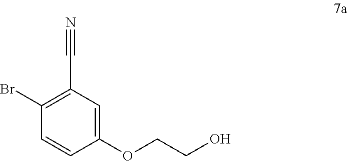 [SMILES: N#Cc1cc(OCCO)ccc1Br]

A mixture of 2-bromo-5-hydroxybenzonitrile (1.98 g, 10 mmol), 2-bromoethanol (1.5 g, 12 mmol,) and K₂CO₃ (2.07 g, 15 mmol) in acetonitrile (15 mL) was stirred at 80° C. overnight. After the reaction was completed, the mixture was filtered and the solid was washed with EtOAc (10 mL) twice. The combined filtrate was concentrated in vacuo and the residue was purified by column chromatography on silica gel (elution with PE: EtOAc 10:1˜1:10) to give 2-bromo-5-(2-hydroxyethoxy)benzonitrile (1.6 g, 66.1% yield) as a white solid.

### Step 2: Preparation of cis-3-[2-[4-(8-chloro-2-quinolyl)-3-cyano-phenoxy]ethoxy]cyclobutanecarboxylic Acid

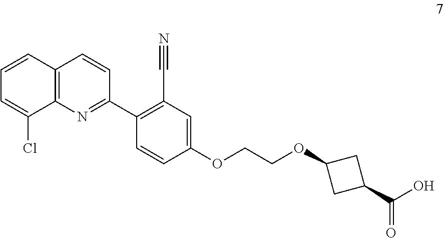 [SMILES: N#Cc1cc(OCCO[C@H]2C[C@@H](C(=O)O)C2)ccc1-c1ccc2cccc(Cl)c2n1]

Example 7 was prepared in analogy to the procedure described for the preparation of Example 1 by using 2-bromo-5-(2-hydroxyethoxy)benzonitrile instead of 2-(4-bromophenoxy)ethanol in Step 1. ¹H NMR (METHANOL-d₄, 400 MHz): δ ppm 8.31 (d, J=8.68 Hz, 1H), 7.86 (d, J=8.56 Hz, 2H), 7.75-7.81 (m, 2H), 7.40-7.47 (m, 1H), 7.36 (d, J=2.69 Hz, 1H), 7.26 (dd, J=8.80, 2.69 Hz, 1H), 4.11 (dd, J=5.26, 3.79 Hz, 2H), 3.87-3.96 (m, 1H), 3.62-3.69 (m, 2H), 2.52-2.64 (m, 1H), 2.38-2.49 (m, 2H), 2.01-2.14 (m, 2H). MS obsd. (ESI⁺) [(M+H)⁺]: 423.2.

### Example 8

### 2-[4-[7-(trifluoromethyl)-2-quinolyl]phenoxy]ethanol

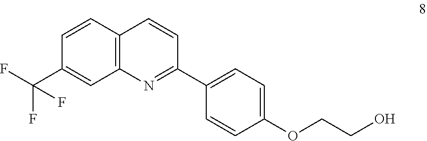 [SMILES: OCCOc1ccc(-c2ccc3ccc(C(F)(F)F)cc3n2)cc1]

A mixture of 2-bromo-7-(trifluoromethyl)quinoline (CAS #: 1352443-02-7, Cat. #: SY046186, from Accela ChemBio Inc, 200 mg, 724 μmol), 2-(4-(4,4,5,5-tetramethyl-1,3,2-dioxaborolan-2-yl)phenoxy)ethanol (191 mg, 724 μmol), Pd(Ph₃P)₄ (25.1 mg, 21.7 μmol) and Cs₂CO₃ (354 mg, 1.09 mmol) in the mixed solvent of toluene (5 mL) and water (1 mL) was charged with N₂ and stirred at 100° C. under microwave condition for 3 hours. After the reaction was completed, the mixture was diluted with water (10 mL) and extracted with EtOAc (25 mL) three times. The combined organic layer was washed with brine, dried over anhydrous Na₂SO₄ and concentrated in vacuo. The residue was purified by column chromatography on silica gel (elution with PE: EtOAc 10:1˜2:1) to give 2-[4-[7-(trifluoromethyl)-2-quinolyl]phenoxy]ethanol (210 mg, 85.2%) as a light grey solid. ¹H NMR (METHANOL-d₄, 400 MHz): δ ppm 8.46 (d, J=8.68 Hz, 1H), 8.39 (s, 1H), 8.18-8.23 (m, 2H), 8.11-8.17 (m, 2 H), 7.77 (dd, J=8.50, 1.65 Hz, 1H), 7.14-7.20 (m, 2H), 4.15-4.21 (m, 2H), 3.92-3.98 (m, 2 H). MS obsd. (ESI⁺) [(M+H)⁺]: 333.5.

### Example 9

### Cis-methyl 3-[2-[4-[7-(trifluoromethyl)-2-quinolyl]phenoxy]ethoxy]cyclobutanecarboxylate

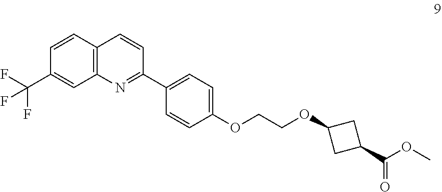 [SMILES: COC(=O)[C@H]1C[C@@H](OCCOc2ccc(-c3ccc4ccc(C(F)(F)F)cc4n3)cc2)C1]

### Step 1: Preparation of 2-[4-[7-(trifluoromethyl)-2-quinolyl]phenoxy]ethyl trifluoromethanesulfonate

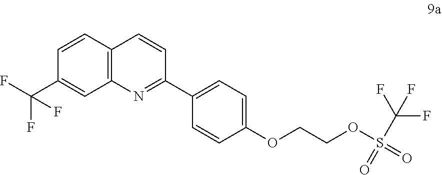 [SMILES: O=S(=O)(OCCOc1ccc(-c2ccc3ccc(C(F)(F)F)cc3n2)cc1)C(F)(F)F]

To a solution of 2-[4-[7-(trifluoromethyl)-2-quinolyl]phenoxy]ethanol (100 mg, 300 μmol) and 2,6-lutidine (38.6 mg, 42 μL, 360 μmol) in DCM (5 mL) was added trifluoromethanesulfonic anhydride (102 mg, 60.9 μL, 360 μmol) dropwise at 0° C. and the mixture was stirred at 0° C. for another 30 minutes. After the reaction was completed, the mixture was diluted with EtOAc (30 mL) and washed with water (10 mL), 0.5 N HCl (10 mL), water (10 mL) and brine (10 mL) in sequence. The organic layer was separated out, dried over anhydrous Na₂SO₄ and concentrated in vacuo to give the crude of 2-[4-[7-(trifluoromethyl)-2-quinolyl]phenoxy]ethyl trifluoromethanesulfonate (140 mg, 100% yield) as a yellow oil, which was used in the next step directly without further purification. MS obsd. (ESI⁺) [(M+H)⁺]: 465.1.

### Step 2: Preparation of cis-methyl 3-[2-[4-[7-(trifluoromethyl)-2-quinolyl]phenoxy]ethoxy]cyclobutanecarboxylate

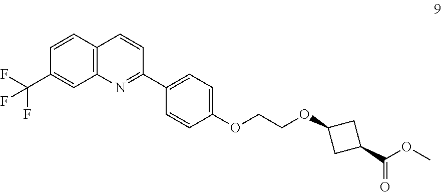 [SMILES: COC(=O)[C@H]1C[C@@H](OCCOc2ccc(-c3ccc4ccc(C(F)(F)F)cc4n3)cc2)C1]

To a mixture of cis-methyl 3-hydroxycyclobutanecarboxylate (58.6 mg, 450 μmol) and NaH (7.21 mg, 300 μmol) in THF (5 mL) cooled at 0° C. was added 2-[4-[7-(trifluoromethyl)-2-quinolyl]phenoxy]ethyl trifluoromethanesulfonate (140 mg, 300 μmol). The mixture was then stirred at 0° C. for another 1 hour. After the reaction was completed, the reaction was quenched with water (20 mL). The resulting mixture was extracted with EtOAc (30 mL) twice. The combined organic layer was washed with brine(10 mL), dried over anhydrous Na₂SO₄ and concentrated in vacuo. The residue was purified by column chromatography on silica gel (elution with PE: EtOAc 10:1˜2:1) to give cis-methyl 3-[2-[4-[7-(trifluoromethyl)-2-quinolyl]phenoxy]ethoxy]cyclobutanecarboxylate (74 mg, 54.3% over 2 steps) as a light yellow solid. ¹H NMR (METHANOL-d₄, 400 MHz): δ ppm 8.45 (d, J=8.68 Hz, 1H), 8.39 (s, 1H), 8.17-8.23 (m, 2H), 8.10-8.16 (m, 2H), 7.76 (dd, J=8.56, 1.71 Hz, 1H), 7.12-7.18 (m, 2H), 4.21 (dd, J=5.38, 3.79 Hz, 2H), 3.98-4.12 (m, 1H), 3.79 (dd, J=5.38, 3.79 Hz, 2H), 3.69 (s, 3H), 2.67-2.82 (m, 1H), 2.50-2.63 (m, 2H), 2.12-2.26 (m, 2H). MS obsd. (ESI⁺) [(M+H)⁺]: 445.6.

### Example 10

### 2-(3-bromophenyl)-7-(trifluoromethyl)quinoline-4-carboxylic acid

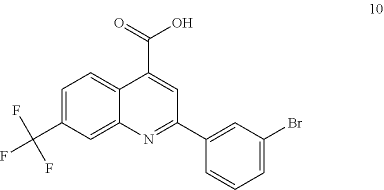 [SMILES: O=C(O)c1cc(-c2cccc(Br)c2)nc2cc(C(F)(F)F)ccc12]

To a solution of 6-(trifluoromethyl)indoline-2,3-dione (100 mg, 465 μmol) and 1-(3-bromophenyl)ethanone (111 mg, 558 μmol) in ethanol (2 mL) was added KOH (78.2 mg, 1.39 mmol) and the mixture was then stirred at 80° C. overnight. After the reaction was completed, the mixture was adjusted to pH ˜6 by addition of HOAc. The resulting solution was then concentrated in vacuo and the residue was purified by preparative HPLC to give 2-(3-bromophenyl)-7-(trifluoromethyl)quinoline-4-carboxylic acid (3.8 mg, 9.5 μmol, 2.04% yield). ¹H NMR (DMSO-d₆, 400 MHz): δ ppm 8.89-8.96 (m, 1H), 8.60-8.67 (m, 1H), 8.52-8.58 (m, 2H), 8.31-8.38 (m, 1H), 7.94-8.04 (m, 1H), 7.74-7.82 (m, 1H), 7.57 (s, 1H). MS obsd. (ESI⁺) [(M+H)⁺]:395.0.

### Example 11

### 2-(3-bromophenyl)-4-methyl-7-(trifluoromethyl)quinoline

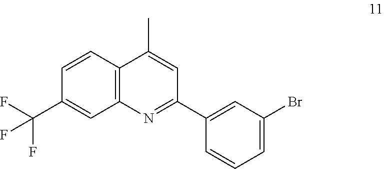 [SMILES: Cc1cc(-c2cccc(Br)c2)nc2cc(C(F)(F)F)ccc12]

A mixture of 1-(2-amino-4-(trifluoromethyl)phenyl)ethanone (100 mg, 492 μmol), 1-(3-bromophenyl)ethanone (98 mg, 492 μmol) and indium (III) chloride (21.8 mg, 98.4 μmol) was stirred at 100° C. for 4 hours. After the reaction was completed, the mixture was partitioned between EtOAc (20 mL) and water (15 mL), the organic layer was then separated out and the aquatic phase was extracted with EtOAc (15 mL) twice. The combined organic layer was washed with brine, dried over Na₂SO₄ and concentrated in vacuo. The residue was then triturated in MeOH (3 mL) and the mixture was then filtered. The solid was collected and dried in vacuo to give 2-(3-bromophenyl)-4-methyl-7-(trifluoromethyl)quinoline (80 mg 43.9% yield). ¹H NMR (DMSO-d₆, 400 MHz): δ ppm 8.51 (t, J=1.76 Hz, 1H), 8.41 (s, 1H), 8.29-8.36 (m, 2H), 8.26 (s, 1H), 7.88 (dd, J=1.76, 8.53 Hz, 1H), 7.73 (ddd, J=0.75, 1.88, 7.91 Hz, 1H), 7.54 (t, J=7.91 Hz, 1H), 2.81 (s, 3H). MS obsd. (ESI⁺) [(M+H)⁺]:336.1.

### Example 12

### 3-[2-[4-[8-chloro-4-(trifluoromethyl)-2-quinolyl]phenoxy]ethoxy]cyclobutanecarboxylic acid

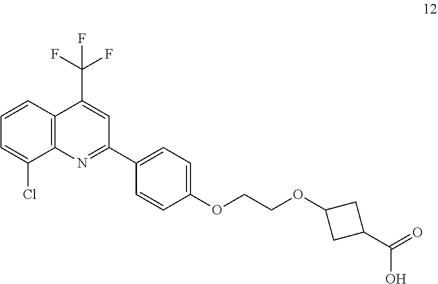 [SMILES: O=C(O)C1CC(OCCOc2ccc(-c3cc(C(F)(F)F)c4cccc(Cl)c4n3)cc2)C1]

### Step 1: Preparation of N-(2-chlorophenyl)-4,4,4-trifluoro-3-oxo-butanamide

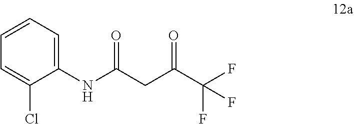 [SMILES: O=C(CC(=O)C(F)(F)F)Nc1ccccc1Cl]

A mixture of 2-chloroaniline (4.0 g, 31.36 mmol) and methyl 4,4,4-trifluoro-3-oxo-butanoate (10.67 g, 62.71 mmol) in water (1 mL) was stirred at 130° C. for 16 hours. The mixture was poured into water (200 mL) and extracted with EtOAc (500 mL) twice. The combined organic phase was dried over Na₂SO₄ and concentrated in vacuo to give the crude of N-(2-chlorophenyl)-4,4,4-trifluoro-3-oxo-butanamide (7.0 g, 83.9% yield) as yellow oil, which was used in the next step directly without further purification. MS obsd. (ESI⁺) [(M+H)⁺]:266.1.

### Step 2: Preparation of 8-chloro-4-(trifluoromethyl)-1H-quinolin-2-one

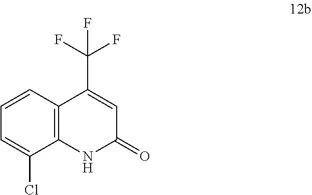 [SMILES: O=c1cc(C(F)(F)F)c2cccc(Cl)c2[nH]1]

A mixture of N-(2-chlorophenyl)-4,4,4-trifluoro-3-oxo-butanamide (7.0 g, 26.35 mmol) in H₂SO₄ (70 mL) was stirred at 90° C. for 16 hours. Then the mixture was poured into water (200 mL) and the resulting suspension was filtered. The solid was collected and dried in vacuo to give 8-chloro-4-(trifluoromethyl)-1H-quinolin-2-one (2.5 g, 38.0% yield) as a white solid, which was used in the next step directly without further purification. MS obsd. (ESI⁺) [(M+H)⁺]:248.1.

### Step 3: Preparation of 2,8-dichloro-4-(trifluoromethyl)quinoline

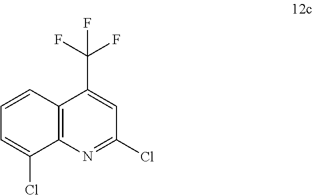 [SMILES: FC(F)(F)c1cc(Cl)nc2c(Cl)cccc12]

A mixture of 8-chloro-4-(trifluoromethyl)-1H-quinolin-2-one (2.5 g, 10.1 mmol) in POCl₃ (20.0 mL, 228.3 mmol) was stirred at 90° C. for 3 hours. After the reaction was completed, the mixture was concentrated in vacuo to give the crude product of 2,8-dichloro-4-(trifluoromethyl)quinoline (2 g, 74.4% yield) as a brown oil, which was used in the next step directly without further purification. MS obsd. (ESI⁺) [(M+H)⁺]:266.0.

### Step 4: Preparation of 4-[8-chloro-4-(trifluoromethyl)-2-quinolyl]phenol

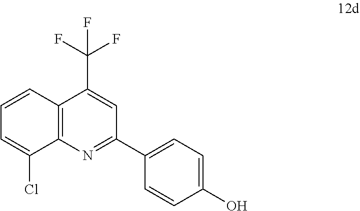 [SMILES: Oc1ccc(-c2cc(C(F)(F)F)c3cccc(Cl)c3n2)cc1]

To a mixture of 2,8-dichloro-4-(trifluoromethyl)quinoline (1.0 g, 3.76 mmol) in 1,4-dioxane (20 mL) and water (2 mL) was added (4-hydroxyphenyl)boronic acid (0.43 g, 3.11 mmol), Pd(PPh₃)₄(0.22 g, 0.190 mmol) and K₂CO₃ (0.78 g, 5.64 mmol). The mixture was charged with N₂ and stirred at 80° C. for 16 hours. Then the mixture was poured into water (30 mL) and the resulting solution was adjusted to pH 4˜5 by addition of concentrated hydrochloric acid. The mixture was then extracted with EtOAc (50 mL) twice. The combined organic phase was dried over Na₂SO₄ and concentrated in vacuo to give the crude product of 4-[8-chloro-4-(trifluoromethyl)-2-quinolyl]phenol (1 g, 82.1% yield) as a yellow solid. which was used in the next step directly without further purification. MS obsd. (ESI⁺) [(M+H)⁺]:324.1.

### Step 5: Preparation of 2-benzyloxyethoxy(trimethyl)silane

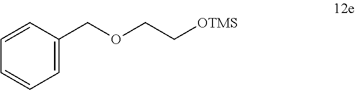

To a solution of 2-benzyloxyethanol (20.0 g, 131.4 mmol) and TEA (20.0 g, 197.1 mmol) in dichloromethane (200 mL) cooled at 0° C. was added trimethylsilyl chloride (17.1 g, 157.7 mmol) and the mixture was then stirred at 25° C. for 16 hours. After the reaction was completed, the mixture was concentrated in vacuo and the residue was purified by column chromatography on silica gel (elution with PE:EtOAc=50:1 to 10:1) to give the 2-benzyloxyethoxy(trimethyl)silane (25.0 g, 84.9%) as a colorless oil.

### Step 6: Preparation of methyl 3-(2-benzyloxyethoxy)cyclobutanecarboxylate

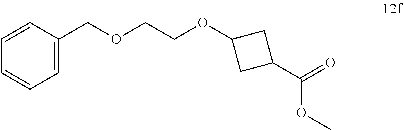 [SMILES: COC(=O)C1CC(OCCOCc2ccccc2)C1]

To a solution of 2-benzyloxyethoxy(trimethyl)silane (25.0 g, 111.4 mmol) and methyl 3-oxocyclobutanecarboxylate (CAS #: 4934-99-0, Cat. #: PB01390, from PharmaBlock (Nanjing) R&D Co. Ltd, 15.0 g, 117.0 mmol) in dichloromethane (200 mL) was added trimethylsilyl trifluoromethanesulfonate (12.4 g, 55.7 mmol) dropwise at −78° C. After addition, the mixture was stirred at −78° C. for additional 1 hour and then to the resulting mixture was added triethylsilane (14.25 g, 122.57 mmol). After addition, the resulting mixture was warmed to room temperature and stirred at room temperature for 1 hour. After the reaction was completed, the mixture was washed with saturated NH₄Cl solution, brine, dried over anhydrous sodium sulfate, and concentrated in vacuo. The residue was purified by column chromatography on silica gel (elution with PE/EtOAc=100:1-˜50:1) to give methyl 3-(2-benzyloxyethoxy)cyclobutanecarboxylate (28 g, 95.1%) as a colorless oil. MS obsd. (ESI⁺) [(M+H)⁺]: 265.1.

### Step 7: Preparation of methyl 3-(2-hydroxyethoxy)cyclobutanecarboxylate

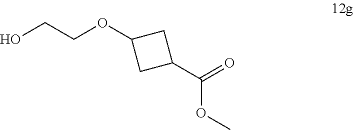

To a solution of methyl 3-(2-benzyloxyethoxy)cyclobutanecarboxylate (28.0 g, 105.9 mmol) in MeOH (300.0 mL) was added Pd(OH)₂(wet) (1.48 g, 10.6 mmol) at room temperature and the mixture was then hydrogenated under H₂ atmosphere at room temperature overnight. After the reaction was completed, the reaction was filtered through silica gel pad and the filtrate was concentrated in vacuo to give 18 g crude methyl 3-(2-hydroxyethoxy)cyclobutanecarboxylate (18 g, 97.6%) as a colorless oil.

### Step 8: Preparation of methyl 3-[2-(p-tolylsulfonyloxy)ethoxy]cyclobutanecarboxylate

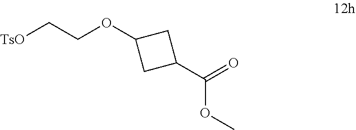 [SMILES: COC(=O)C1CC(OCCOS(=O)(=O)c2ccc(C)cc2)C1]

To a solution of methyl 3-(2-hydroxyethoxy)cyclobutanecarboxylate (5 g, 28.7 mmol) and DMAP (5.26 g, 43.1 mmol) in dichloromethane (80 mL) was added 4-methylbenzene-1-sulfonyl chloride (6.02 g, 31.6 mmol) at room temperature and the mixture was then stirred at room temperature overnight. After the reaction was completed, the mixture was washed with 1N HCl (25 mL), water (15 mL), saturated NaHCO₃ solution, brine and concentrated in vacuo to give the crude methyl 3-[2-(p-tolylsulfonyloxy)ethoxy]cyclobutanecarboxylate (8.1 g, 85.6%) as a colorless oil, which was used in next step directly without further purification. MS obsd. (ESI⁺) [(M+H)⁺]: 329.2.

### Step 9: Preparation of methyl 3-[2-[4-[8-chloro-4-(trifluoromethyl)-2-quinolyl]phenoxy]ethoxy]cyclobutanecarboxylate

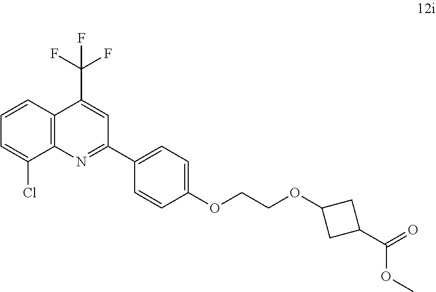 [SMILES: COC(=O)C1CC(OCCOc2ccc(-c3cc(C(F)(F)F)c4cccc(Cl)c4n3)cc2)C1]

To a mixture of 4-[8-chloro-4-(trifluoromethyl)-2-quinolyl]phenol (300.0 mg, 0.930 mmol) and methyl 3-[2-(p-tolylsulfonyloxy)ethoxy]cyclobutanecarboxylate (334.77 mg, 1.02 mmol) in DMF (5 mL) was added K₂CO₃ (192.13 mg, 1.39 mmol) and the mixture was then stirred at 80° C. for 16 hours. After the reaction was completed, the mixture was poured into water (20 mL) and the resulting suspension was extracted with EtOAc (30 mL) three times. The combined organic phase was dried over Na₂SO₄ and concentrated in vacuo to give the crude of methyl 3-[2-[4-[8-chloro-4-(trifluoromethyl)-2-quinolyl]phenoxy]ethoxy]cyclobutanecarboxylate (400 mg, 89.5% yield) as a yellow solid which was used in next step directly without further purification. MS obsd. (ESI⁺) [(M+H)⁺]: 480.2.

### Step 10: Preparation of 3-[2-[4-[8-chloro-4-(trifluoromethyl)-2-quinolyl]phenoxy]ethoxy]cyclobutanecarboxylic acid

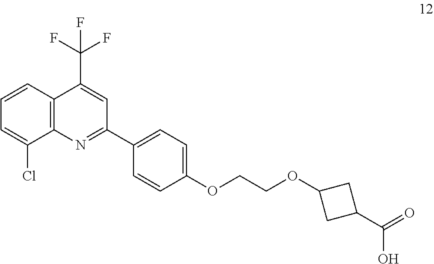 [SMILES: O=C(O)C1CC(OCCOc2ccc(-c3cc(C(F)(F)F)c4cccc(Cl)c4n3)cc2)C1]

To a mixture of methyl 3-[2-[4-[8-chloro-4-(trifluoromethyl)-2-quinolyl]phenoxy]ethoxy]cyclobutanecarboxylate (400.0 mg, 0.830 mmol) in the mixed solvent of THF (5 mL) and water (1 mL) was added LiOH.H₂O (104.93 mg, 2.5 mmol) and the mixture was stirred at room temperature for 3 hours. After the reaction was completed, the mixture was poured into water (30 mL) and adjusted to pH 4˜5 by addition of concentrated hydrochloric acid. The resulting solution was extracted with EtOAc (30 mL) three times. The combined organic layer was washed with brine, dried over Na₂SO₄ and concentrated in vacuo. The residue was then triturated in EtOAc (10 mL) and the mixture was then filtered. The solid was collected and dried in vacuo to give 3-[2-[4-[8-chloro-4-(trifluoromethyl)-2-quinolyl]phenoxy]ethoxy]cyclobutanecarboxylic acid (150 mg, 38.8% yield) as a white solid. ¹H NMR (DMSO-d₆, 400 MHz): δ ppm 12.18 (s, 1H), 8.53 (s, 1H), 8.40-8.42 (d, J=8.8 Hz, 2H), 8.10-8.12 (d, J=7.6 Hz, 1H), 8.02-8.04 (d, J=8.0 Hz, 1H), 7.70-7.74 (m, 1H), 7.15-7.17 (d, J=8.8 Hz, 2H), 3.69-4.11 (m, 3H), 3.68 (m, 2H), 2.44-2.94 (m, 1H), 2.40-2.42 (m, 2H), 2.05-2.19 (m, 2H). MS obsd. (ESI+) [(M+H)+]:466.1.

### Example 13

### 2-[2-[4-[8-chloro-4-(trifluoromethyl)-2-quinolyl]phenoxy]ethoxy]acetic acid

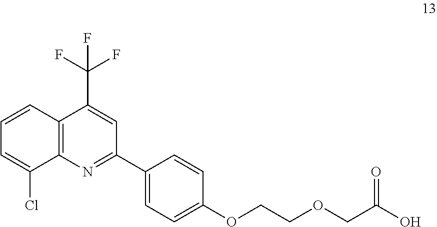 [SMILES: O=C(O)COCCOc1ccc(-c2cc(C(F)(F)F)c3cccc(Cl)c3n2)cc1]

### Step 1: Preparation of ethyl 2-(2-hydroxyethoxy)acetate

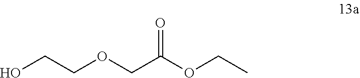

To a solution of ethyl 2-(2-benzyloxyethoxy)acetate (2.0 g, 8.39 mmol) in ethanol (20 mL) was added palladium hydroxide (1.18 g, 8.39 mmol) and the mixture was then hydrogenated under H₂ atmosphere at room temperature overnight. After the reaction was completed, the reaction was filtered through silica gel pad and the filtrate was concentrated in vacuo to give ethyl 2-(2-hydroxyethoxy)acetate (1.1 g, 7.42 mmol, 44.23% yield) as a colorless oil.

### Step 2: Preparation of ethyl 2-[2-(p-tolylsulfonyloxy)ethoxy]acetate

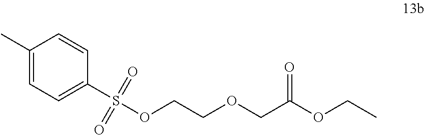 [SMILES: CCOC(=O)COCCOS(=O)(=O)c1ccc(C)cc1]

To a solution of ethyl 2-(2-hydroxyethoxy)acetate (1.1 g, 7.42 mmol) in DCM (20 mL) at room temperature was added triethylamine (2.07 mL, 14.85 mmol), m-toluenesulfonyl chloride (1.7 g, 8.91 mmol) and the reaction mixture was stirred at room temperature for 12 hours. After the reaction was completed, the mixture was concentrated in vacuo and the residue was purified by column chromatography on silica gel (elution with PE: EtOAc=50:1˜10:1) to give ethyl 2-[2-(p-tolylsulfonyloxy)ethoxy]acetate (0.400 g, 1.32 mmol) as a colorless oil. MS obsd. (ESI⁺) [(M+H)⁺]: 303.1.

### Step 3: Preparation of 2-[2-[4-[8-chloro-4-(trifluoromethyl)-2-quinolyl]phenoxy]ethoxy]acetic acid

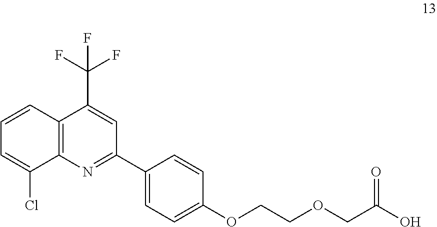 [SMILES: O=C(O)COCCOc1ccc(-c2cc(C(F)(F)F)c3cccc(Cl)c3n2)cc1]

Example 13 was prepared in analogy to the procedure described for the preparation of Example 12 by using ethyl 2-[2-(p-tolylsulfonyloxy)ethoxy]acetate instead of methyl 3-[2-(p-tolylsulfonyloxy)ethoxy]cyclobutanecarboxylate in Step 9.

Example 13: ¹H NMR (DMSO-d₆, 400 MHz): δ ppm 8.55 (s, 1H), 8.42-8.44 (d, J=8.8 Hz, 2H), 8.11-8.13 (d, J=7.6 Hz, 1H), 8.04-8.06 (d, J=8.0 Hz, 1H), 7.71-7.75 (m, 1H), 7.15-7.18 (d, J=9.2 Hz, 2H), 4.25-4.26 (m, 2H), 4.13-4.24 (m, 2H), 3.86-3.89 (m, 2H). MS obsd. (ESI⁺) [(M+H)⁺]: 426.1.

### Example 14

### 3-[[4-(8-chloro-4-methoxy-2-quinolyl)phenoxy]methyl]benzoic acid

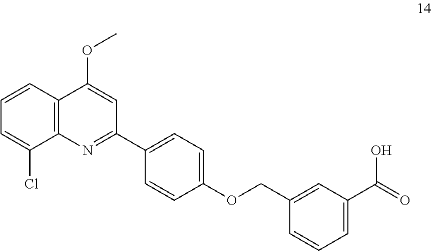 [SMILES: COc1cc(-c2ccc(OCc3cccc(C(=O)O)c3)cc2)nc2c(Cl)cccc12]

### Step 1: Preparation of [4-[(2-acetyl-6-chloro-phenyl)carbamoyl]phenyl] acetate

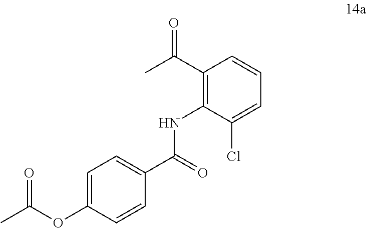 [SMILES: CC(=O)Oc1ccc(C(=O)Nc2c(Cl)cccc2C(C)=O)cc1]

To a solution of 1-(2-amino-3-chloro-phenyl)ethanone (3.0 g, 17.7 mmol) and TEA (3.4 g, 34.0 mmol) in DCM (20 mL) cooled at 0° C. was added the solution of (4-chlorocarbonylphenyl) acetate (3.4 g, 17.7 mg) in DCM (20 mL) dropwise. After addition, the mixture was stirred at 50° C. overnight. After the reaction was completed, the mixture was concentrated in vacuo. The residue was purified by preparative HPLC to give [4-[(2-acetyl-6-chloro-phenyl)carbamoyl]phenyl] acetate (2.2 g, 37.4% yield) as a white solid. MS obsd. (ESI⁺) [(M+H)⁺]: 332.1.

### Step 2: Preparation of N-(2-acetyl-6-chloro-phenyl)-4-hydroxy-benzamide

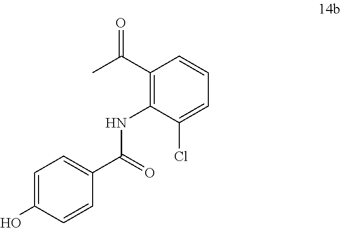 [SMILES: CC(=O)c1cccc(Cl)c1NC(=O)c1ccc(O)cc1]

To a solution of [4-[(2-acetyl-6-chloro-phenyl)carbamoyl]phenyl] acetate (1.0 g, 3.0 mmol) in acetonitrile (20 mL) was added K₂CO₃ (3.2 g, 9.0 mmol) and the mixture was then stirred at room temperature overnight. After the reaction was completed, the mixture was adjusted to pH ˜4 by addition of 2 N HCl and the resulting solution was extracted with EtOAc (20 mL) three times. The combined organic layer was dried over Na₂SO₄ and concentrated in vacuo to give the crude product of N-(2-acetyl-6-chloro-phenyl)-4-hydroxy-benzamide (0.9 g) as a yellow oil, which was used in the next step directly without further purification. MS obsd. (ESI⁺) [(M+H)⁺]: 290.1.

### Step 3: Preparation of methyl 3-[[4-[(2-acetyl-6-chloro-phenyl)carbamoyl]phenoxy]methyl]benzoate

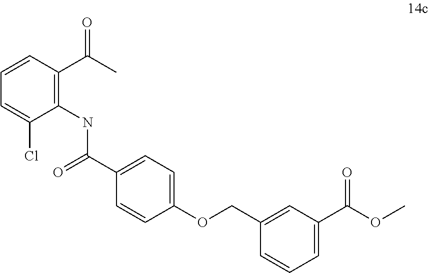 [SMILES: COC(=O)c1cccc(COc2ccc(C(=O)Nc3c(Cl)cccc3C(C)=O)cc2)c1]

To a solution of N-(2-acetyl-6-chloro-phenyl)-4-hydroxy-benzamide (0.9 g, 3.1 mmol) and K₂CO₃ (1.8 g, 6.8 mmol) in acetone (20.0 mL) was added methyl 3-(bromomethyl)benzoate (780.0 mg, 3.4 mmol) and the mixture was then stirred at 60° C. for 10 hours. After the reaction was completed, the mixture was concentrated in vacuo and the residue was redissolved in DCM (30 mL). The resulting suspension was filtered and the filtrate was concentrated in vacuo to give the crude product of methyl 3-[[4-[(2-acetyl-6-chloro-phenyl)carbamoyl]phenoxy]methyl]benzoate (1.0 g) as a yellow oil. MS obsd. (ESI⁺) [(M+H)⁺]: 438.2.

### Step 4: Preparation of 3-[[4-(8-chloro-4-oxo-1H-quinolin-2-yl)phenoxy]methyl]benzoic acid

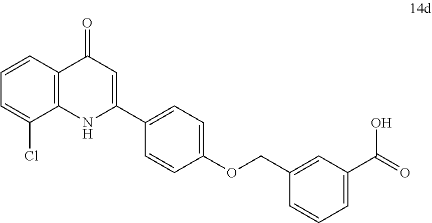 [SMILES: O=C(O)c1cccc(COc2ccc(-c3cc(=O)c4cccc(Cl)c4[nH]3)cc2)c1]

To a solution of methyl 3-[[4-[(2-acetyl-6-chloro-phenyl)carbamoyl]phenoxy]methyl]benzoate (1.0 g, 2.3 mmol) in the mixed solvent of dioxane (20 mL) and water (2.0 mL) was added NaOH (1.0 g, 23.0 mmol) and the mixture was stirred at room temperature for 10 hours.

After the reaction was completed, the mixture was filtered and the filtrate was concentrated in vacuo. The residue was then triturated in the mixed solvent of EtOAc (5 mL) and MeOH (5 mL) and the mixture was then filtered. The solid was collected and dried in vacuo to afford 3-[[4-(8-chloro-4-oxo-1H-quinolin-2-yl)phenoxy]methyl]benzoic acid (20.0 mg) as a yellow solid. ¹H NMR (DMSO-d₆, 400 MHz): δ ppm 13.04 (br. s., 1H), 11.75 (br. s., 1H), 10.53 (br. s., 1H), 8.16 (d, J=8.5 Hz, 1H), 8.04-8.12 (m, 1H), 7.90 (dd, J=14.9, 7.7 Hz, 1H), 7.69-7.81 (m, 1H), 7.50-7.61 (m, 1H), 7.38-7.47 (m, 1H), 7.21 (d, J=8.8 Hz, 1H), 6.35 (s, 1H), 5.27-5.36 ppm (m, 1H). MS obsd. (ESI⁺) [(M+H)⁺]: 405.9.

### Step 5: Preparation of methyl 3-[[4-(8-chloro-4-methoxy-2-quinolyl)phenoxy]methyl]benzoate

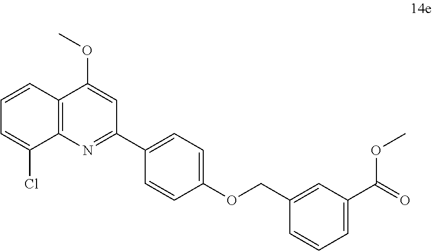 [SMILES: COC(=O)c1cccc(COc2ccc(-c3cc(OC)c4cccc(Cl)c4n3)cc2)c1]

To a mixture of 3-[[4-(8-chloro-4-oxo-1H-quinolin-2-yl)phenoxy]methyl]benzoic acid (20.0 mg, 49.3 μmol) in DMF (2 mL) was added NaH (5.0 mg, 125.0 μmol) and the mixture was stirred at 80° C. for 1 hour. Then to the mixture was added Me₂SO₄ (4.3 mg, 24.6 μmol) and the mixture was stirred at 80° C. for another 2 hours. After the reaction was completed, the mixture was quenched by addition of 0.5M HCl (10 mL) and the resulting solution was extracted by THF (20 mL). The organic layer was washed with brine, dried over Na₂SO₄ and concentrated in vacuo. The residue was then triturated in the mixed solvent of MeOH (2 mL) and water (2 mL) and the mixture was then filtered. The solid was collected and dried in vacuo to give methyl 3-[[4-(8-chloro-4-methoxy-2-quinolyl)phenoxy]methyl]benzoate (10 mg, 46.7% yield) as a yellow solid. ¹H NMR (DMSO-d₆, 400 MHz): δ ppm8.35-8.36 (d, 2H, J=8 Hz), 8.05-8.09 (m, 2H), 7.90-7.93 (m, 2H), 7.61 (s, 1H), 7.57 (m, 2H), 7.46 (m, 1H), 7.219-7.22 (d, 2H, J=8.8 Hz), 5.31 (s, 2H), 4.16 (s, 3H), 3.86 (s, 3H). MS obsd. (ESI⁺) [(M+H)⁺]: 434.1.

### Step 6: Preparation of 3-[[4-(8-chloro-4-methoxy-2-quinolyl)phenoxy]methyl]benzoic acid

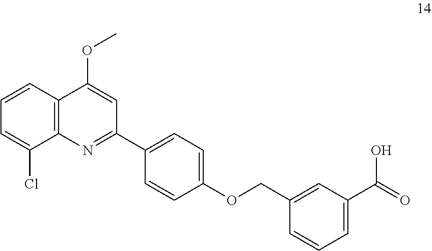 [SMILES: COc1cc(-c2ccc(OCc3cccc(C(=O)O)c3)cc2)nc2c(Cl)cccc12]

A mixture of methyl 3-[[4-(8-chloro-4-methoxy-2-quinolyl)phenoxy]methyl]benzoate (10.0 mg, 23.0 μmol) and NaOH (5.0 mg, 125.0 μmol) in the mixed solvent of DMSO (3 mL) and water (1.0 mL) was stirred at room temperature for 1 hour. After the reaction was completed, the mixture was adjusted to pH˜6 by addition of 1 M HCl and the resulting solution was purified by preparative HPLC (TFA) to give 3-[[4-(8-chloro-4-methoxy-2-quinolyl)phenoxy]methyl]benzoic acid (5 mg) as yellow solid. ¹H NMR (DMSO-d₆, 400 MHz): δ ppm 8.18-8.20 (d, 1H, J=8.1 Hz), 8.14-8.16 (m, 3H), 7.94-7.99 (m, 2H), 7.53 (m, 1H), 7.50-7.51 (m, 3H), 7.20-7.23 (m, 2H), 5.28 (s, 2H), 4.26 (s, 3H). MS obsd. (ESI⁺) [(M+H)⁺]: 434.1.

### Example 15

### Cis-3-[2-[4-(8-chloro-3-methoxy-2-quinolyl)phenoxy]ethoxy]cyclobutanecarboxylic acid

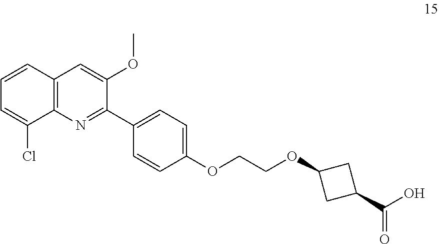 [SMILES: COc1cc2cccc(Cl)c2nc1-c1ccc(OCCO[C@H]2C[C@@H](C(=O)O)C2)cc1]

### Step 1: Preparation of 8-chloro-3-methoxy-quinolin-2-ol

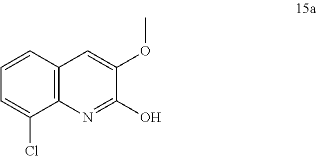

A mixture of 7-chloroindoline-2,3-dione (1 g, 5.51 mmol), (trimethylsilyl)diazomethane (5.51 mL, 11 mmol) and diethylamine (806 mg, 896 μL, 11 mmol) in ethanol (25 mL) was stirred at room temperature. After 18 hours, the resulting mixture was concentrated in vacuo. The residue was purified by column chromatography on silica gel (elution with PE:EtOAc 10:1 to 2:1) to give 8-chloro-3-methoxy-quinolin-2-ol (0.48 g, 41.6%) as a dark brown solid. MS obsd. (ESI⁺) [(M+H)⁺]: 210.0.

### Step 2: Preparation of (8-chloro-3-methoxy-2-quinolyl) trifluoromethanesulfonate

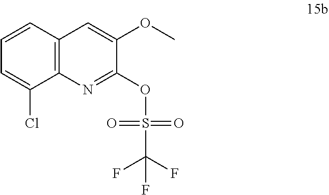 [SMILES: COc1cc2cccc(Cl)c2nc1OS(=O)(=O)C(F)(F)F]

To a solution of 8-chloro-3-methoxy-quinolin-2-ol (0.6 g, 2.86 mmol) and 2,6-lutidine (399 mg, 433 μL, 3.72 mmol) in DCM (20 mL) cooled at 0° C. was added trifluoromethanesulfonic anhydride (1.05 g, 626 μL, 3.72 mmol) dropwise and the mixture was kept at 0° C. for 30 mins. After the reaction was completed, the reaction solution was diluted with EtOAc (100 mL) and washed with water (20 mL), 0.5 N HCl (20 mL) and brine (20 mL) in sequence. The organic layer was dried over anhydrous Na₂SO₄ and concentrated in vacuo, the residue was purified by column chromatography on silica gel (elution with PE: EtOAc 100:10 to 100:50) to give (8-chloro-3-methoxy-2-quinolyl) trifluoromethanesulfonate (0.7 g, 71.6%) as a white oil. MS obsd. (ESI⁺) [(M+H)⁺]:342.0.

### Step 3: Preparation of 2-[4-(8-chloro-3-methoxy-2-quinolyl)phenoxy]ethanol

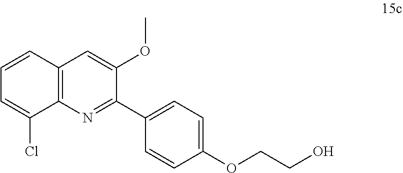 [SMILES: COc1cc2cccc(Cl)c2nc1-c1ccc(OCCO)cc1]

A mixture of (8-chloro-3-methoxy-2-quinolyl) trifluoromethanesulfonate (400 mg, 1.17 mmol), 2-(4-(4,4,5,5-tetramethyl-1,3,2-dioxaborolan-2-yl)phenoxy)ethanol (340 mg, 1.29 mmol), Pd(Ph₃P)₄ (67.6 mg, 58.5 μmol) and Cs₂CO₃ (458 mg, 1.4 mmol) in toluene (15 mL) and water (3 mL) was charged with N₂ and then stirred at 100° C. for 16 hours. After cooling, the mixture was diluted with EtOAc (50 mL) and washed with water (10 mL). The organic layer was separated out and concentrated in vacuo, the residue was purified by column chromatography on silica gel (elution with PE: EtOAc 100:10 to 100:50) to give 2-[4-(8-chloro-3-methoxy-2-quinolyl)phenoxy]ethanol (0.27 g, 70.5%) as an off-white oil. MS obsd. (ESI⁺) [(M+H)⁺]:330.0.

### Step 4: Preparation of cis-3-[2-[4-(8-chloro-3-methoxy-2-quinolyl)phenoxy]ethoxy]cyclobutanecarboxylic acid

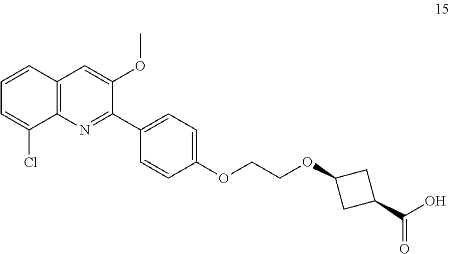 [SMILES: COc1cc2cccc(Cl)c2nc1-c1ccc(OCCO[C@H]2C[C@@H](C(=O)O)C2)cc1]

Example 15 was prepared in analogy to the procedure described for the preparation of Example 1 by using 2-[4-(8-chloro-3-methoxy-2-quinolyl)phenoxy]ethanol instead of 2-(4-(8-chloroquinolin-2-yl)phenoxy)ethanol in Step 4. ¹H NMR (DMSO-d₆, 400 MHz): δ ppm 8.07 (s, 2H), 7.96 (s, 1H), 7.84-7.93 (m, 1H), 7.70-7.77 (m, 1H), 7.46-7.55 (m, 1H), 6.99-7.15 (m, 2H), 4.10-4.20 (m, 2H), 4.00 (s, 4H), 3.62-3.72 (m, 2H), 2.56-2.63 (m, 1H), 2.41-2.46 (m, 2H), 1.95-2.08 (m, 2H). MS obsd. (ESI⁺) [(M+H)⁺]: 428.2.

### Example 16

### 3-[2-[[5,8-dichloro-2-(4-hydroxyphenyl)-3-quinolyl]oxy]ethoxy]cyclobutanecarboxlic acid

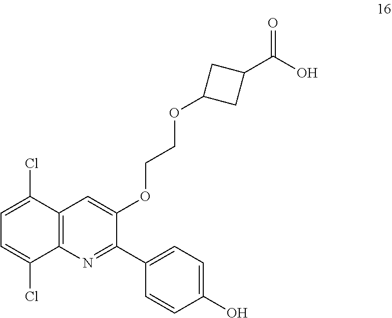 [SMILES: O=C(O)C1CC(OCCOc2cc3c(Cl)ccc(Cl)c3nc2-c2ccc(O)cc2)C1]

### Step 1: Preparation of (5,8-dichloro-3-methoxy-2-quinolyl) trifluoromethanesulfonate

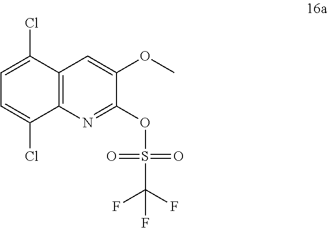 [SMILES: COc1cc2c(Cl)ccc(Cl)c2nc1OS(=O)(=O)C(F)(F)F]

Compound 16a was prepared in analogy to the procedure described for the preparation of compound 15b by using 4,7-dichloroindoline-2,3-dione instead of 7-chloroindoline-2,3-dione in Step 1.

### Step 2: Preparation of 2-(4-benzyloxyphenyl)-5,8-dichloro-3-methoxy-quinoline

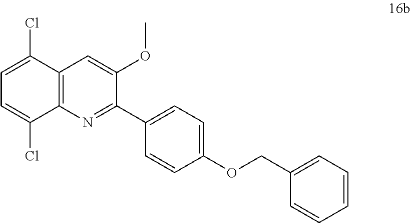 [SMILES: COc1cc2c(Cl)ccc(Cl)c2nc1-c1ccc(OCc2ccccc2)cc1]

A mixture of (5,8-dichloro-3-methoxy-2-quinolyl) trifluoromethanesulfonate (200 mg, 532 μmol), 2-(4-(benzyloxy)phenyl)-4,4,5,5-tetramethyl-1,3,2-dioxaborolane (181 mg, 585 μmol), Pd(Ph₃P)₄ (30.7 mg, 26.6 μmol) and Cs₂CO₃ (208 mg, 638 μmol) in toluene (15 mL) and water (3 mL) was charged with N₂ and then stirred at 100° C. for 16 hours. After cooling, the mixture was partitioned between EtOAc (50 mL) and water (10 mL). The organic layer was separated out and concentrated in vacuo, the residue was purified by column chromatography on silica gel (elution with PE: EtOAc 100:10 to 100:50) to give 2-(4-benzyloxyphenyl)-5,8-dichloro-3-methoxy-quinoline (200 mg, 91.7%) as an off-white solid. MS obsd. (ESI⁺) [(M+H)⁺]:411.1.

### Step 3: Preparation of 5,8-dichloro-2-(4-hydroxyphenyl)quinolin-3-ol

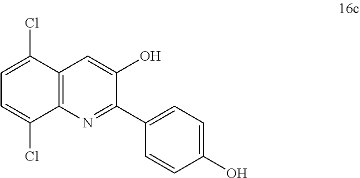 [SMILES: Oc1ccc(-c2nc3c(Cl)ccc(Cl)c3cc2O)cc1]

A mixture of 2-(4-benzyloxyphenyl)-5,8-dichloro-3-methoxy-quinoline (200 mg, 487 μmol) and tribromoborane (122 mg, 487 μmol) in DCM (2 mL) was stirred at 25° C. for 14 hours. After the reaction was completed the mixture was partitioned between EtOAc (50 mL) and water (10 mL). The organic layer was separated out, dried over Na₂SO₄ and concentrated in vacuo to give the crude of 5,8-dichloro-2-(4-hydroxyphenyl)quinolin-3-ol (120 mg, 80.4%) as a brown solid, which was used in the next step directly without further purification. MS obsd. (ESI⁺) [(M+H)⁺]:307.1.

### Step 4: Preparation of methyl 3-[2-[[5,8-dichloro-2-(4-hydroxyphenyl)-3-quinolyl]oxy]ethoxy]cyclobutanecarboxylate

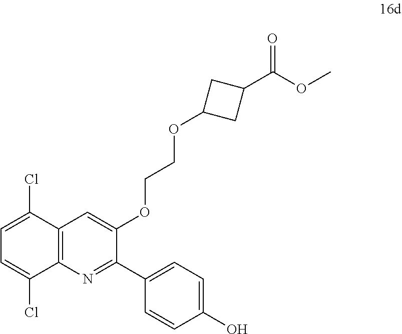 [SMILES: COC(=O)C1CC(OCCOc2cc3c(Cl)ccc(Cl)c3nc2-c2ccc(O)cc2)C1]

A mixture of 5,8-dichloro-2-(4-hydroxyphenyl)quinolin-3-ol (120 mg, 392 μmol), methyl 3-(2-(tosyloxy)ethoxy)cyclobutanecarboxylate (129 mg, 392 μmol) and potassium carbonate (163 mg, 1.18 mmol) in DMF (6 mL) was stirred at 80° C. for 2 hours. After the reaction was completed, the mixture was quenched with water (20 mL) and extracted with DCM (25 mL) three times, the combined organic layer was dried over Na₂SO₄ and concentrated in vacuo. The residue was purified by column chromatography on silica gel (elution with PE: EtOAc 100:10 to 100:50) to give methyl 3-[2-[[5,8-dichloro-2-(4-hydroxyphenyl)-3-quinolyl]oxy]ethoxy]cyclobutanecarboxylate (140 mg, 77%) as a brown solid. MS obsd. (ESI⁺) [(M+H)⁺]: 462.1.

### Step 5: Preparation of 3-[2-[[5,8-dichloro-2-(4-hydroxyphenyl)-3-quinolyl]oxy]ethoxy]cyclobutanecarboxylic acid

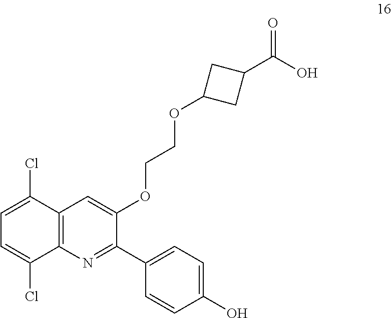 [SMILES: O=C(O)C1CC(OCCOc2cc3c(Cl)ccc(Cl)c3nc2-c2ccc(O)cc2)C1]

A mixture of methyl 3-[2-[[5,8-dichloro-2-(4-hydroxyphenyl)-3-quinolyl]oxy]ethoxy]cyclobutanecarboxylate (140 mg, 303 μmol) and lithium hydroxide (36.3 mg, 1.51 mmol) in MeOH (5 mL) was stirred at room temperature for 48 hours. After the reaction was completed, the mixture was concentrated in vacuo. The residue was suspended in 1N HCl (15 mL) and the resulting suspension was extracted with EtOAc(30 mL) three times. The combined organic phase was washed with brine, dried over Na₂SO₄ and concentrated in vacuo. The residue was purified by preparative HPLC to give 3-[2-[[5,8-dichloro-2-(4-hydroxyphenyl)-3-quinolyl]oxy]ethoxy]cyclobutanecarboxylic acid (35 mg, 24.5%) as a light yellow solid. ¹H NMR (MeOH-d₄, 400 MHz): δ ppm 8.09 (d, J=8.80 Hz, 2H), 7.66 (s, 1H), 7.48 (d, J=8.07 Hz, 1H), 7.36 (d, J=8.19 Hz, 1H), 6.79 (d, J=8.68 Hz, 2H), 4.17 (m, 3H), 3.63-3.72 (m, 2H), 2.90-2.99 (m, 1H), 2.38-2.47 (m, 2H), 2.02-2.25 (m, 2H). MS obsd. (ESI⁺) [(M+H)⁺]: 449.1.

### BIOLOGICAL EXAMPLES

### Example 17: Engineered HepDES19 Primary Screen Assay

The assay was employed to screen for novel cccDNA inhibitors. HepDES19 is a cccDNA-producing cell line. In this cell line, HBeAg in the cell culture supernatant as surrogate marker, as HBeAg production depends on cccDNA level and activity. HepDES19 is an engineered cell line which contains a 1.1 unit length HBV genome, and pgRNA transcription from the transgene is controlled by Tetracycline (Tet). In the absence of Tet, pgRNA transcription will be induced, but HBV e antigen (HBeAg) could not be produced from this pgRNA due to very short leader sequence before the HBeAg start codon and the start codon is disrupted. Only after cccDNA is formed, the missing leader sequence and start codon mutation would be restored from the 3′-terminal redundancy of pgRNA, and then HBeAg could be synthesized. Therefore, HBeAg could be used as a surrogate marker for cccDNA (Zhou, T. et al., Antiviral Res.(2006), 72(2), 116-124; Guo, H. et al., J. Virol. (2007), 81(22), 12472-12484).

HepDES19 cells were seeded at 2×10⁶ cells per T150 flask and cultured with the culture medium (Dulbecco's Modified Eagle Medium: Nutrient Mixture F-12 [DMEM-F12, Gibco Cat. 11320-82], 10% Fetal Bovine Serum [FBS, Clontech Cat. 631101], 0.1 mM Non-Essential Amino Acids Solution [NEAA, Gibco Cat. 11140-050], 50 μg/mL Penicillin-Streptomycin [PS, Invitrogen Cat. 15140-163], 500 μg/mL Geneticin [G418, Invitrogen Cat. 10131-027]) containing 3 μg/mL Tet (Sigma, Cat. 87128) for 5 days. Cells were then seeded at 4×10⁶ cells per T150 in the same culture medium as described above in the absence of Tet for 8 days. Cells were then harvested and frozen at density of 2×10⁶ cells per mL. For compound testing, the frozen cells were thawed and seeded into 96-well plates at a density of 6×10⁴ cells per well. At 24 hrs after seeding, half log serial dilutions of compounds made with Dimethyl sulfoxide (DMSO, Sigma, Cat. D2650) were further diluted with the same culture medium as described above before they were added to the cells to reach desired final compound concentrations and 1% DMSO concentration. Plates were then incubated at 37° C. for another 5 days before measurement of HBeAg level and cell viability. Intracellular HBeAg level were measured with enzyme-linked immunosorbent assay (ELISA) kit (Shanghai Kehua Diagnostic Medical Products Co., Ltd). Cell viability was assessed using Cell Counting Kit-8 (Donjindo, Cat. CK04-20). IC₅₀ values were derived from the dose-response curve using 4 parameter logistic curve fit method.

The compounds of the present invention were tested for their capacity to inhibit extracellular HBeAg level as described herein. The compounds of this invention were found to have IC₅₀ below 50 μM. Particular compounds of formula (I) were found to have IC₅₀ below 5.0 μM. Results of HepDES19 primary screen assay are given in Table 1.

TABLE 1
Activity data in HepDES19
primary screen assay

| Example No. | IC₅₀ (μM) |
|---|---|
| 1 | 0.008 |
| 2 | 0.233 |
| 3 | 0.141 |
| 4 | 2.117 |
| 5 | 3.910 |
| 6 | 0.537 |
| 7 | 18.384 |
| 8 | 18.477 |
| 9 | 17.809 |
| 10 | 19.514 |
| 11 | 2.074 |
| 12 | 0.515 |
| 13 | 1.673 |
| 14 | 1.855 |
| 15 | 2.532 |
| 16 | 3.615 |

### Example 18: Cryopreserved Primary Human Hepatocytes (PHH) Assay

This assay is used to confirm the anti-HBV effect of the compounds in HBV PHH infection assay. Cryopreserved PHH (BioreclamationIVT, Lot YJM) was thawed at 37° C. and gently transferred into pre-warmed InVitroGRO HT medium (BioreclamationIVT, Cat. S03317). The mixture was centrifuged at 70 relative centrifugal force (RCF) for 3 minutes at RT, and the supernatant was discarded. Pre-warmed InVitroGRO CP medium (BioreclamationIVT, Cat #S03316) was added to the cell pellet to gently re-suspend cells. The cells were seeded at the density of 5.8×10⁴ cells per well to collagen I coated 96-well plate (Gibco, Cat. A1142803) with the InVitroGRO CP medium. All plates were incubated at 37° C. with 5% CO₂ and 85% humidity.

At 20 hours after plating, the medium was changed to PHH culture medium (Dulbecco's Modified Eagle Medium (DMEM)/F12 (1:1) (Gibco, Cat. 11320-033), 10% fetal bovine serum (Gibco Cat. 10099141), 100 U/mL penicillin, 100 μg/mL streptomycin (Gibco, Cat. 151401-122), 5 ng/mL human epidermal growth factor (Invitrogen Cat. PHG0311L), 20 ng/mL dexamethasone (Sigma, Cat. D4902) and 250 ng/mL human recombinant insulin (Gibco, Cat. 12585-014)). And the cells were incubated at 37° C. with 5% CO₂ and 85% humidity for 4 hours. The medium was then changed to pre-warmed PHH culture medium containing 4% polyethylene glycol (PEG) MW8000 (Sigma, Cat. P1458-50ML) and 1% DMSO (Sigma, Cat. D2650). 5.8×10⁶ genomic equivalents of HBV were added into the medium.

At 24 hours post-infection, the cells were gently washed with PBS and refreshed with PHH culture medium supplemented with 1% DMSO, and 0.25 mg/mL Matrix gel (Corning, Cat. 356237) at 200 μL per well. All plates were immediately placed in at 37° C. CO₂ incubator. 24 hours later, serial dilutions of compounds made with DMSO were further diluted with the same culture medium (PHH culture medium supplemented with 1% DMSO and 0.25 mg/mL Matrix gel as described above) before they were added to the cells to reach desired final compound concentrations and 1% DMSO concentration. The medium containing the compounds were refreshed every three days.

At 9 days post-compound treatment, extracellular HBsAg level were measured with Chemiluminescence Immuno Assay (CLIA) kit (Autobio, HBsAg Quantitative CLIA). Extracellular HBV DNA was extracted by MagNA Pure 96 system (Roche) and then determined by quantitative PCR with the following primers and probe:

HBV-Forward Primer (SEQ ID NO:1): AAGAAAAACCCCGCCTGTAA (5′ to 3′);

HBV-Reverse Primer (SEQ ID NO:2): CCTGTTCTGACTACTGCCTCTCC(5′ to 3′);

HBV-Probe: 5′+tetramethylrhodamine+SEQ ID NO:3+black hole quencher 2-3′, wherein SEQ ID NO:3 is CCTGATGTGATGTTCTCCATGTTCAGC.

HBsAg IC₅₀ and HBV DNA IC₅₀ values were derived from the dose-response curve using 4 parameter logistic curve fit method. Results of Cryopreserved PHH assay are given in Table 2.

TABLE 2
HBsAg IC₅₀ data in
Cryopreserved PHH assay

| Example | HBsAg IC₅₀ |
|---|---|
| No. | (μM) |
| 1 | 0.015 |
| 2 | 0.970 |
| 3 | 11.268 |
| 4 | 6.768 |
| 5 | 2.952 |
| 6 | 8.345 |
| 11 | 1.656 |
| 12 | 3.311 |
| 13 | 5.503 |

1. A compound of formula (I), 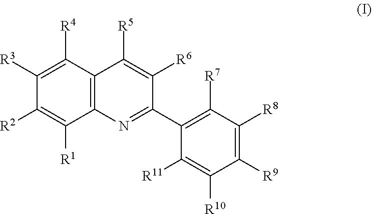
wherein:
R¹ is H, halogen, cyano, C₁₋₆alkyl or haloC₁₋₆alkyl;
R² is H, halogen, cyano, C₁₋₆alkyl or haloC₁₋₆alkyl;
R³ is H, halogen, cyano, C₁₋₆alkyl or haloC₁₋₆alkyl;
R⁴ is H, halogen, cyano, C₁₋₆alkyl or haloC₁₋₆alkyl;
R⁵ is H, halogen, haloC₁₋₆alkyl, C₁₋₆alkyl, carboxy, C₁₋₆alkoxy, C₁₋₆alkoxyC₁₋₆alkyl, C₁₋₆alkoxycarbonyl, C₁₋₆alkyl-C(O)O—C₁₋₆alkyl-, hydroxyC₁₋₆alkyl or hydroxy;
R⁶ is H, C₁₋₆alkoxy, (carboxyC₃₋₇cycloaIkoxy)C₁₋₆alkoxy or (C₁₋₆alkoxycarbonyl-C₃₋₇cycloaIkoxy)C₁₋₆alkoxy;
R⁷ is H, halogen, cyano, C₁₋₆alkyl or haloC₁₋₆alkyl;
R⁸ is H, halogen, cyano, C₁₋₆alkyl or haloC₁₋₆alkyl;
R⁹ is H, hydroxy, C₁₋₆aIkoxy, hydroxyC₁₋₆alkoxy, (carboxyC₁₋₆alkoxy)C₁₋₆alkoxy, carboxyphenylC₁₋₆alkoxy, (carboxyC₃₋₇cycloalkoxy)C₁₋₆alkoxy or (C₁₋₆alkoxycarbonylC₃₋₇cycloalkoxy)C₁₋₆alkoxy;
R¹⁰ is H, halogen, cyano, C₁₋₆alkyl or haloC₁₋₆alkyl; and
R¹¹ is H, halogen, cyano, C₁₋₆alkyl or haloC₁₋₆alkyl;
with the proviso that R¹ and R² are not H simultaneously; or that R⁸ and R⁹ are not H simultaneously;
or a pharmaceutically acceptable salt thereof.

2. A compound according to claim 1, wherein:
R¹ is H or halogen;
R² is H or haloC₁₋₆alkyl;
R³ is H;
R⁴ is H or halogen;
R⁵ is H, C₁₋₆alkyl, carboxy, C₁₋₆alkoxy or haloC₁₋₆alkyl;
R⁶ is H, C₁₋₆alkoxy or (carboxyC₃₋₇cycloalkoxyC₁₋₆alkoxy;
R⁷ is H or cyano;
R⁸ is H, halogen or haloC₁₋₆alkyl;
R⁹ is H, hydroxy, C₁₋₆alkoxy, hydroxyC₁₋₆alkoxy, (carboxyC₁₋₆alkoxy)C₁₋₆alkoxy, carboxyphenylC₁₋₆alkoxy, (carboxyC₃₋₇cycloalkoxy)C₁₋₆alkoxy or (C₁₋₆alkoxycarbonyl-C₃₋₇cycloalkoxy)C₁₋₆alkoxy;
R¹⁰ is H; and
R¹¹ is H;
with the provisos that R¹ and R² are not H simultaneously; or that R⁸ and R⁹ are not H simultaneously;
or a pharmaceutically acceptable salt thereof.

3. A compound according to claim 2, wherein:
R¹ is H or chloro;
R² is H or CF₃;
R³ is H;
R⁴ is H or chloro;
R⁵ is H, methyl, methoxy, carboxy or CF₃;
R⁶ is H, methoxy or (carboxycyclobutoxy)ethoxy;
R⁷ is H or cyano;
R⁸ is H, bromo or CF₃;
R⁹ is H, hydroxy, methoxy, (carboxycyclobutoxy)ethoxy, (carboxymethoxy)ethoxy, hydroxyethoxy, (carboxyphenyl)methoxy or (methoxycarbonylcyclobutoxy)ethoxy;
R¹⁰ is H; and
R¹¹ is H;
with the proviso that R¹ and R² are not H simultaneously; or that R⁸ and R⁹ are not H simultaneously;
or a pharmaceutically acceptable salt thereof.

4. A compound according to claim 1, or a pharmaceutically acceptable salt thereof, wherein R⁵ is H or haloC₁₋₆alkyl.

5. A compound according to claim 4, or a pharmaceutically acceptable salt thereof, wherein R⁵ is H or CF₃.

6. A compound according to claim 1, or a pharmaceutically acceptable salt thereof, wherein R⁸ is H or haloC₁₋₆alkyl.

7. A compound according to claim 6, or a pharmaceutically acceptable salt thereof, wherein R⁸ is H or CF₃.

8. A compound according to claim 1, or a pharmaceutically acceptable salt thereof, wherein R⁹ is H, hydroxyC₁₋₆alkoxy, (carboxyC₁₋₆-alkoxy)C₁₋₆alkoxy or (carboxyC₃₋₇cycloalkoxy)C₁₋₆alkoxy.

9. A compound according to claim 8, or a pharmaceutically acceptable salt thereof, wherein R⁹ is H, (carboxycyclobutoxy)ethoxy, (carboxymethoxy)ethoxy or hydroxyethoxy.

10. A compound according to claim 1, wherein:
R¹ is halogen;
R² is H;
R³ is H;
R⁴ is H or halogen;
R⁵ is H or haloC₁₋₆alkyl;
R⁶ is H, C₁₋₆alkoxy or (carboxyCv₇cycloalkoxy)C₁₋₆alkoxy;
R⁷ is H;
R⁸ is H or haloC₁₋₆alkyl;
R⁹ is hydroxy, (carboxyC₁₋₆alkoxy)C₁₋₆alkoxy or (carboxyC₃₋₇cycloalkoxy)C₁₋₆alkoxy;
R¹⁰ is H; and
R¹¹ is H;
or a pharmaceutically acceptable salt thereof.

11. A compound according to claim 10, wherein:
R¹ is chloro;
R² is H;
R³ is H;
R⁴ is H or chloro;
R⁵ is H or CF₃;
R⁶ is H, methoxy or (carboxycyclobutoxy)ethoxy;
R⁷ is H;
R⁸ is H or CF₃;
R⁹ is hydroxy, (carboxymethoxy)ethoxy or (carboxycyclobutoxy)ethoxy;
R¹⁰ is H; and
R¹¹ is H;
or a pharmaceutically acceptable salt thereof.

12. A compound according to claim 1, or a pharmaceutically acceptable salt thereof, with the proviso that R¹ and R² are not H simultaneously and that R⁸ and R⁹ are not H simultaneously.

13. A compound according to claim 2, selected from:
cis-3-[2-[4-(8-chloro-2-quinolyl)phenoxy]ethoxy]cyclobutanecarboxylic acid;
2-[2-[4-(8-chloro-2-quinolyl)phenoxy]ethoxy] acetic acid;
8-chloro-2-[4-methoxy-3-(trifluoromethyl)phenyl]quinoline;
2-[4-(8-chloro-2-quinolyl)-2-(trifluoromethyl)phenoxy]ethanol;
cis-3-[2-[4-(8-chloro-2-quinolyl)-2-(trifluoromethyl)phenoxy]ethoxy]cyclobutanecarboxylic acid; 2-[2-[4-(8-chloro-2-quinolyl)-2-(trifluoromethyl)phenoxy]ethoxy]acetic acid;
cis-3-[2-[4-(8-chloro-2-quinolyl)-3-cyano-phenoxy]ethoxy]cyclobutanecarboxylic acid;
2-[4-[7-(trifluoromethyl)-2-quinolyl]phenoxy]ethanol;
cis-methyl 3-[2-[4-[7-(trifluoromethyl)-2-quinolyl]phenoxy]ethoxy]cyclobutanecarboxylate;
2-(3-bromophenyl)-7-(trifluoromethyl)quinoline-4-carboxylic acid;
2-(3-bromophenyl)-4-methyl-7-(trifluoromethyl)quinolone;
3-[2-[4-[8-chloro-4-(trifluoromethyl)-2-quinolyl]phenoxy]ethoxy]cyclobutanecarboxylic acid;
2-[2-[4-[8-chloro-4-(trifluoromethyl)-2-quinolyl]phenoxy]ethoxy]acetic acid;
3-[[4-(8-chloro-4-methoxy-2-quinolyl)phenoxy]methyl]benzoic acid; and
cis-3-[2-[4-(8-chloro-3-methoxy-2-quinolyl)phenoxy]ethoxy]cyclobutanecarboxylic acid and 3-[2-[[5,8-dichloro-2-(4-hydroxyphenyl)-3-quinolyl]oxy]ethoxy]cyclobutanecarboxylic acid;
or a pharmaceutically acceptable salt thereof.

14. A compound according to claim 13, selected from:
cis-3-[2-[4-(8-chloro-2-quinolyl)phenoxy]ethoxy]cyclobutanecarboxylic acid;
2-[2-[4-(8-chloro-2-quinolyl)phenoxy]ethoxy] acetic acid;
cis-3-[2-[4-(8-chloro-2-quinolyl)-2-(trifluoromethyl)phenoxy]ethoxy]cyclobutanecarboxylic acid; 2-[2-[4-(8-chloro-2-quinolyl)-2-(trifluoromethyl)phenoxy]ethoxy]acetic acid;
3-[2-[4-[8-chloro-4-(trifluoromethyl)-2-quinolyl]phenoxy]ethoxy]cyclobutanecarboxylic acid;
2-[2-[4-[8-chloro-4-(trifluoromethyl)-2-quinolyl]phenoxy]ethoxy]acetic acid;
cis-3-[2-[4-(8-chloro-3-methoxy-2-quinolyl)phenoxy]ethoxy]cyclobutanecarboxylic acid; and
3-[2-[[5,8-dichloro-2-(4-hydroxyphenyl)-3-quinolyl]oxy]ethoxy]cyclobutanecarboxylic acid;
or a pharmaceutically acceptable salt thereof.

15. A process for the preparation of a compound according to claim 1 comprising any one of the following steps:
(a) Hydrolysis of a compound of formula (IX), 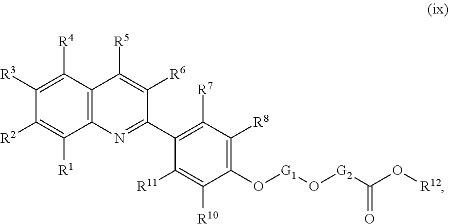
in the presence of a base;
(b) Suzuki crossing coupling reaction of quinoline (VI), 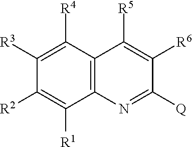
(VI), with borate ester (V) in the presence of a catalyst;
(c) Substitution of trifluoromethanesulfonate(VIII), 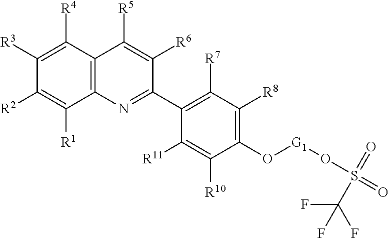
(VIII), with the compound of formula (XI) in the presence of a base;
(d) Suzuki crossing coupling reaction of quinoline (VI), 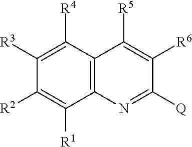
(VI), with boronic acid (XII) in the presence of a catalyst;
(e) Substitution of trifluoromethanesulfonate(XIII), 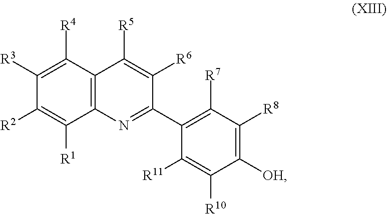
the compound of formula (XIV) in the presence of a base;
(f) Condensation of compound of formula (XV), 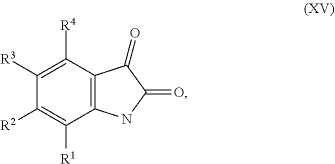
the compound of formula (XVI) in the presence of a base;
wherein R¹, R², R³, R⁴, R⁵, R⁶, R⁷, R⁸, R⁹, R¹⁰ and R¹¹ are defined in claim 1; R¹² is C₁₋₆alkyl; Gi is C₁₋₆alkyl; G₂ is C₁₋₆alkyl or C₃₋₇cycloalkyl; Q is halogen or OTf.

16. (canceled)

17. A pharmaceutical composition comprising a compound in accordance with claim 1 and a therapeutically inert carrier.

18. (canceled)

19. (canceled)

20. The use of a compound according to claim 1 for the inhibition of HBV cccDNA.

21. The use of a compound according to claim 1 to 14 for the inhibition of HBeAg.

22. The use of a compound according to claim 1 to 14 for the inhibition of HBsAg.

23. The use of a compound according to claim 1 to 14 for the inhibition of HBV DNA.

24. A compound according to claim 1 to 14 for the treatment or prophylaxis of HBV infection.

25. A compound according to when manufactured according to the process of claim 15.

26. A method for the treatment or prophylaxis of HBV infection, which method comprises administering to a patient in need thereof an effective amount of a compound as defined in claim 1.
